SEQUENCE LISTING (31 sequences)

SEQ ID NO: 1 - DNA, 21 bp, Artificial
  Oligonucleotide
gatggaagagtgtggagagtg

SEQ ID NO: 2 - DNA, 22 bp, Artificial
  Oligonucleotide
cagtctcgattatgcacattcc

SEQ ID NO: 3 - DNA, 20 bp, Artificial
  Oligonucleotide
gtgatcaggtgtaaggctgc

SEQ ID NO: 4 - DNA, 19 bp, Artificial
  Oligonucleotide
ctgccttcagcttcgtgac

SEQ ID NO: 5 - DNA, 21 bp, Artificial
  Oligonucleotide
tcttgacgagttcttctgagg

SEQ ID NO: 6 - DNA, 22 bp, Artificial
  Oligonucleotide
cagttgactgtcaatgaactgg

SEQ ID NO: 7 - DNA, 1613 bp, Homo sapiens
gggcatgggcgcggccaccccggatgtcagccccccgcgccgaccagaatccgtgaacatggcgaacgaggttatcaagtgcaaggctgcagttgcttgggaggctggaaagcctctctccatagaggagatagaggtggcacccccaaaggctcatgaagttcgaatcaagatcattgccactgcggtttgccacaccgatgcctataccctgagtggagctgatcctgagggttgttttccagtgatcttgggacatgaaggtgctggaattgtggaaagtgttggtgagggagttactaagctgaaggcgggtgacactgtcatcccactttacatcccacagtgtggagaatgcaaattttgtctaaatcctaaaactaacctttgccagaagataagagtcactcaagggaaaggattaatgccagatggtaccagcagatttacttgcaaaggaaagacaattttgcattacatgggaaccagcacattttctgaatacacagttgtggctgatatctctgttgctaaaatagatcctttagcacctttgtataaagtctgccttctaggttgtggcatttcaaccggttatggtgctgctgtgaacactgccaagttggagcctggctctgtttgtgccgtctttggtctgggaggagtcggattggcagttatcatgggctgtaaagtggctggtgcttcccggatcattggtgtggacatcaataaagataaatttgcaagggccaaagagtttggagccactgaatgtattaaccctcaggatttaagtaaacccatccaggaagtgctcattgagatgaccgatggaggagtggactattcctttgaatgtattggtaatgtgaaggtcatgagagcagcacttgaggcatgtcacaagggctggggcgtcagcgtcgtggttggagtagctgcttcaggtgaagaaattgccactcgtccattccagctggtaacaggtcgcacatggaaaggcactgcctttggaggatggaagagtgtagaaagtgtcccaaagttggtgtctgaatatatgtccaaaaagataaaagttgatgaatttgtgactcacaatctgtcttttgatgaaatcaacaaagcctttgaactgatgcattctggaaagagcattcgaactgttgtaaagatttaattcaaaagagaaaaataatgtccatcctgtcgtgatgtgataggagcagcttaacaggcagggagaagcgcctccaacctcacagcctcgtagagcttcacagctactccagaaaatagggttatgtgtgtcattcatgaatctctataatcaaggacaaggataattcagtcatgaacctgttttctggatgctcctccacataaataattgctagttataaggatatttaacataataaaagtaattctacattgtgtgaattgtcttgtttatgctgtcatcattgtcacggtttgtctgcccattatcttcattctgcaagggaaagggaaaggaagcagggcagtggtgggtgtctgaaacctcagaaacataacgttgaacttttaagggtctcagtccccgttgattaaagaacagatccccg

SEQ ID NO: 8 - DNA, 2496 bp, Homo sapiens
agcggcacgcaccacagctcgagcaccgcccttccggttggagccattgcaagccccccccacgccccgcccccctcgctaggcgctcgccacgcccatgcctccgtcgctgcgcggcccaccccggatgtcagccccccgcgccgaccagaatccgtgaacatggcgaacgaggttatcaagtgcaaggctgcagttgcttgggaggctggaaagcctctctccatagaggagatagaggtggcacccccaaaggctcatgaagttcgaatcaagatcattgccactgcggtttgccacaccgatgcctataccctgagtggagctgatcctgagggttgttttccagtgatcttgggacatgaaggtgctggaattgtggaaagtgttggtgagggagttactaagctgaaggcgggtgacactgtcatcccactttacatcccacagtgtggagaatgcaaattttgtctaaatcctaaaactaacctttgccagaagataagagtcactcaagggaaaggattaatgccagatggtaccagcagatttacttgcaaaggaaagacaattttgcattacatgggaaccagcacattttctgaatacacagttgtggctgatatctctgttgctaaaatagatcctttagcacctttgtataaagtctgccttctaggttgtggcatttcaaccggttatggtgctgctgtgaacactgccaagttggagcctggctctgtttgtgccgtctttggtctgggaggagtcggattggcagttatcatgggctgtaaagtggctggtgcttcccggatcattggtgtggacatcaataaagataaatttgcaagggccaaagagtttggagccactgaatgtattaaccctcaggatttaagtaaacccatccaggaagtgctcattgagatgaccgatggaggagtggactattcctttgaatgtattggtaatgtgaaggtcatgagagcagcacttgaggcatgtcacaagggctggggcgtcagcgtcgtggttggagtagctgcttcaggtgaagaaattgccactcgtccattccagctggtaacaggtcgcacatggaaaggcactgcctttggaggatggaagagtgtagaaagtgtcccaaagttggtgtctgaatatatgtccaaaaagataaaagttgatgaatttgtgactcacaatctgtcttttgatgaaatcaacaaagcctttgaactgatgcattctggaaagagcattcgaactgttgtaaagatttaattcaaaagagaaaataatgtccatcctgtcgtgatgtgataggagcagcttaacaggcagggagaagcgcctccaacctcacagcctcgtagagcttcacagctactccagaaaatagggttatgtgtgtcattcatgaatctctataatcaaggacaaggataattcagtcatgaacctgttttctggatgctcctccacataaataattgctagtttattaaggaatattttaacataataaaagtaatttctacatttgtgtggaaattgtcttgttttatgctgtcatcattgtcacggtttgtctgcccattatcttcattctgcaagggaaagggaaaggaagcagggcagtggtgggtgtctgaaacctcagaaacataacgttgaacttttaagggtctcagtccccgttgattaaagaacagatcctagccatcagtgacaaagttaatcaggacccaagtctgcttctgtgatattatctttaagggaggtactgtgccttgttcatacctgtaccccaaattcctaggatggcatctgccttcagggggcactaaaatgtattattgaaacagcattctgggcttaaataggtgtatgtatgtgttggttgtgactgtactatttctagtatagtgaactacatactgaatatccaagttctcagcacctacttttgtcaaatcttaacattttgccacttcgagatcacattgccattcctcccctccaagaggtaacaattatccacaatttgatgtttatcattcctgtgttgttgtactttcactgtgtataacctaaaccatctactctttagtactgttttatatatttttaagcctcatacttgctcattctacagcttttttcactcattattgtataattatatctgaagctctcgttcattaattttagtcctgtgtagcagaattcaattacgggaactaccataatttatctgttctccagtccagttgaaggcatgaagttgttgccagtttctgtattataacactgtagtggaacattcttctgcattgggctcactgcgtgttacctaagacgtatcacagaataaacacatttagccttatagacattgccaaattgctcttcaaagtaaatgtgagtttttgtgaattacatgagtatggaatggtgttttattatgactttagtttgcattttcctcaa

SEQ ID NO: 9 - DNA, 448 bp, Homo sapiens
tttcagtgaaattcccgttccctcaccgtccacatctgtaatttgcaagtcttatctacgataactgctgtaaagttacacggggaagccctttcccgacaaaaaaaacgagttctgcaacaagtccgtcggattttagcaatgaaaccggcgccgtgcagcctcgccggcgagtgcaccctggaacgcacaacttagcggcacgcaccacagctcgagcaccgcccttccggttggagccattgcaagccccccccacgccccgcccccctcgctaggcgctcgccacgcccatgcctccgtcgctgcgcggcccaccccggatgtcagccccccgcgccgaccagaatccgtgaacatggcgaacgaggtagggcccgttgagcggagggccctgagtccaagggagggagtgcatggcatgggactaggctgctatcctcggggc

SEQ ID NO: 10 - DNA, 146 bp, Homo sapiens
tccccacttcaggttatcaagtgcaaggctgcagttgcttgggaggctggaaagcctctctccatagaggagatagaggtggcacccccaaaggctcatgaagttcgaatcaaggtaatgatacatttaaggcactgggaaaaaaa

SEQ ID NO: 11 - DNA, 383 bp, Homo sapiens
cattcgttgttttaatgtcctgatagatcattgccactgcggtttgccacaccgatgcctataccctgagtggagctgatcctgagggttgttttccagtgatcttgggacatgaaggtgctggaattgtggaaagtgttggtgagggagttactaagctgaaggcgggtaaggagaatacttgaaccacttgtttaataattttggcttattcctatggggaaattgtttttctgataaaactacccactatttatgaataggtatcatctaagtagattgtcaagattaagttgattcctcatctgggaagcacaaatatttggatatttttttctctctgattttgcagactactgtttttgtgaaacttttctaagtac

SEQ ID NO: 12 - DNA, 326 bp, Homo sapiens
ctttgcatttgtttccaggtgacactgtcatcccactttacatcccacagtgtggagaatgcaaattttgtctaaatcctaaaactaacctttgccagaagataaggttagtatctttttatgttcttcttaaaatacaagtgctgcgggataattaaggaatcacagagaccgaggggttgaggaggaattatttaataatttaggttcattaacccagtcggattaacgttcaaaggactgagtcccgaacaaagagtcaagctaccttttaagcatttcgtggggtggggggagacctttgtagggggagcatattacagaag

SEQ ID NO: 13 - DNA, 934 bp, Homo sapiens
tggataagattataaatatcttataaatttcctttataaggcattgctgcaaggtgctaaattactaatgaatatatttgaaattgtagtttacaacactttcttaatatttactggtcattatttttaaaacatttctttttcctgtgggttttaagaaacctaattccaacttgcctttttcctttttttttctttagagtcactcaagggaaaggattaatgccagatggtaccagcagatttacttgcaaaggaaagacaattttgcattacatgggaaccagcacattttctgaatacacagttgtggctgatatctctgttgctaaaatagatcctttagcacctttggataaagtctgccttctaggttgtggcatttcaaccggttatggtgctgctgtgaacactgccaaggtaagagactgacttgggttttttgcttctgccttctaatttaattcagtgaaactttcctggaatagtgaaattggccaatctgtatgaaacccagtgattctcttgactctggtagaatgagtactttataaactttctttactcctagttggagcctggctctgtttgtgccgtctttggtctgggaggagtcggattggcagttatcatgggctgtaaagtggctggtgcttcccggatcattggtgtggacatcaataaagataaatttgcaagggccaaagagtttggagccactgaatgtattaaccctcaggattttagtaaacccatccaggaagtgctcattgagatgaccgatggaggagtggactattcctttgaatgtattggtaatgtgaaggtcatggtgagtatgggcttcattcctttttagtttatggaactgctttttttgaagtgcagttttagattaaaaattgttttgttttggcaaaggaaaagtctctta

SEQ ID NO: 14 - DNA, 307 bp, Homo sapiens
aaatgcactgacttctgtgatttggtcatatctctgtgcctgcagagagcagcacttgaggcatgtcacaagggctggggcgtcagcgtcgtggttggagtagctgcttcaggtgaagaaattgccactcgtccattccagctggtaacaggtcgcacatggaaaggcactgcctttggaggtaattcgatggatgagatgactgcattttctcttttgttagttgcattggcagatgtttaatcccagccacctaattttttttatattaacaagaatcttcatgagttccttctcattataagtt

SEQ ID NO: 15 - DNA, 1519 bp, Homo sapiens
tgtgatttcttttaggatggaagagtgtagaaagtgtcccaaagttggtgtctgaatatatgtccaaaaagataaaagttgatgaatttgtgactcacaatctgtcttttgatgaaatcaacaaagcctttgaactgatgcattctggaaagaggtaggctttctctttatatatcatagaatgtaataatgatgttgagtttgaggggatggagagtcgaaaaacacaattttagttgtcttaaaggtatcatttaaaacttagtatgttgcttccttttacagcattcgaactgttgtaaagatttaattcaaaagagaaaataatgtccatcctgtcgtgatgtgataggagcagcttaacaggcagggagaagcgcctccaacctcacagcctcgtagagcttcacagctactccagaaaatagggttatgtgtgtcattcatgaatctctataatcaaggacaaggataattcagtcatgaacctgttttctggatgctcctccacataaataattgctagtttattaaggaatattttaacataataaaagtaatttctacatttgtgtggaaattgtcttgttttatgctgtcatcattgtcacggtttgtctgcccattatcttcattctgcaagggaaagggaaaggaagcagggcagtggtgggtgtctgaaacctcagaaacataacgttgaacttttaagggtctcagtccccgttgattaaagaacagatcctagccatcagtgacaaagttaatcaggacccaagtctgcttctgtgatattatctttaagggaggtactgtgccttgttcatacctgtaccccaaattcctaggatggcatctgccttcagggggcactaaaatgtattattgaaacagcattctgggcttaaataggtgtatgtatgtgttggttgtgactgtactatttctagtatagtgaactacatactgaatatccaagttctcagcacctacttttgtcaaatcttaacattttgccacttcgagatcacattgccattcctcccctccaagaggtaacaattatccacaatttgatgtttatcattcctgtgttgttgtactttcactgtgtataacctaaaccatctactctttagtactgttttatatatttttaagcctcatacttgctcattctacagcttttttcactcattattgtataattatatctgaagctctcgttcattaattttagtcctgtgtagcagaattcaattacgggaactaccataatttatctgttctccagtccagttgaaggcatgaagttgttgccagtttctgtattataacactgtagtggaacattcttctgcattgggctcactgcgtgttacctaagacgtatcacagaataaacacatttagccttatagacattgccaaattgctcttcaaagtaaatgtgagtttttgtgaattacatgagtatggaatggtgttttattatgactttagtttgcattttcctcaa

SEQ ID NO: 16 - DNA, 1533 bp, Mus musculus
gatcccgaactagcggccatggcgaaccaggtgatcaggtgtaaggctgcagtcgcctgggaggcgggaaagcctctctccatagaggagatagaagtggcccctccaaaggctcatgaagttcggattaagatccttgccactgctgtttgccacaccgatgcctataccctgagcggagctgaccccgaggggtgtttcccagtgatcttgggacatgaaggtgctggaattgtggaaagtgttggtgaaggggtcacgaagctgaaggcaggtgacactgtcatcccactctacatcccacagtgtggagaatgcaagttttgtctgaatcctaaaacaaacctttgccagaaaataagggtcactcaggggaagggattaatgccagatgggactagcagatttacctgcaaaggaaagtctgtttttcacttcatggggactagcacattttccgagtacacagttgtggctgacatctctgttgctaaaatcgatccttcggcccctttggataaagtctgccttctcggctgtggtatttcaactggctacggggctgctgtgaacactgccaaggtggagcctggttctacttgtgccgtctttggcctgggaggagttggactggcagtgatcatgggctgtaaggtggctggtgcatcccggatcattggtatcgacatcaataaagataaattcgcaaaggccaaagaatttggagcctctgaatgtattagcccccaagacttcagtaaatccatccaggaagtcctcgttgagatgacagatgggggcgtggattactcctttgagtgcattggcaacgtgaaggtcatgagatcagcccttgaggcagcccacaaaggctggggtgtcagtgtggtagtgggagtagctgcttcaggggaagaaatctccactcgtccattccagctggtgacaggacgcacatggaaaggcaccgcctttggaggatggaagagtgtggagagtgtcccaaagctggtgtctgaatatatgtccaaaaagataaaagttgacgaatttgtgaccggcaatctctccttcgaccaaattaaccaagcctttgatctgatgcactcgggagacagcattcgaactgttctaaagatgtaagttctgaagagaacactgtccatcctgcactctttcctgtgattggctagaaacagacaggatttacaagctaaccacgtcgtagacttcagaaatgactagaaggaatgtgcataatcgagactgtaatcatgaactaggcaaattccacataaacaattgctgctcattgtgcaacattttaacataataaaaatactttctgcatgtgtgtgtgagttgaacatcttgtcatcttgtaggatcccagttcattgacagtcaactgtgtctagaaacagtgctaacccaaaaatgaaggctgatcttgaattcatccacatgggtttagaatagccgaaatcagaatattctttctttctcagcaaataaattttagaagtgtatact

SEQ ID NO: 17 - protein, 392 aa, Homo sapiens [sequence omitted]

SEQ ID NO: 18 - protein, 374 aa, Homo sapiens
MANEVIKCKAAVAWEAGKPLSIEEIEVAPPKAHEVRIKIIATAVCHTDAYTLSGADPEGCFPVILGHEGAGIVESVGEGVTKLKAGDTVIPLYIPQCGECKFCLNPKTNLCQKIRVTQGKGLMPDGTSRFTCKGKTILHYMGTSTFSEYTVVADISVAKIDPLAPLYKVCLLGCGISTGYGAAVNTAKLEPGSVCAVFGLGGVGLAVIMGCKVAGASRIIGVDINKDKFARAKEFGATECINPQDLSKPIQEVLIEMTDGGVDYSFECIGNVKVMRAALEACHKGWGVSVVVGVAASGEEIATRPFQLVTGRTWKGTAFGGWKSVESVPKLVSEYMSKKIKVDEFVTHNLSFDEINKAFELMHSGKSIRTVVKI

SEQ ID NO: 19 - protein, 374 aa, Homo sapiens
MANEVIKCKAAVAWEAGKPLSIEEIEVAPPKAHEVRIKIIATAVCHTDAYTLSGADPEGCFPVILGHEGAGIVESVGEGVTKLKAGDTVIPLYIPQCGECKFCLNPKTNLCQKIRVTQGKGLMPDGTSRFTCKGKTILHYMGTSTFSEYTVVADISVAKIDPLAPLDKVCLLGCGISTGYGAAVNTAKLEPGSVCAVFGLGGVGLAVIMGCKVAGASRIIGVDINKDKFARAKEFGATECINPQDFSKPIQEVLIEMTDGGVDYSFECIGNVKVMRAALEACHKGWGVSVVVGVAASGEEIATRPFQLVTGRTWKGTAFGGWKSVESVPKLVSEYMSKKIKVDEFVTHNLSFDEINKAFELMHSGKSIRTVVKI

SEQ ID NO: 20 - protein, 374 aa, Mus musculus
MANQVIRCKAAVAWEAGKPLSIEEIEVAPPKAHEVRIKILATAVCHTDAYTLSGADPEGCFPVILGHEGAGIVESVGEGVTKLKAGDTVIPLYIPQCGECKFCLNPKTNLCQKIRVTQGKGLMPDGTSRFTCKGKSVFHFMGTSTFSEYTVVADISVAKIDPSAPLDKVCLLGCGISTGYGAAVNTAKVEPGSTCAVFGLGGVGLAVIMGCKVAGASRIIGIDINKDKFAKAKEFGASECISPQDFSKSIQEVLVEMTDGGVDYSFECIGNVKVMRSALEAAHKGWGVSVVVGVAASGEEISTRPFQLVTGRTWKGTAFGGWKSVESVPKLVSEYMSKKIKVDEFVTGNLSFDQINQAFDLMHSGDSIRTVLKM

SEQ ID NO: 21 - protein, 374 aa, Homo sapiens; identical to SEQ ID NO: 19

SEQ ID NO: 22 - protein, 378 aa, Arabidopsis thaliana
TEGKPIRCKAAILRKAGEPLVIEEIQVDPPQAYEVRIKILCTSLCHTDVTFWKLDSGPLARFPRILGHEAVGVVESIGEKVDGFKQGDVVLPVFHPQCEECKECISPKSNWCTKYTNDYLSNTRRYGMTSRFKDSRGEDIHHFIFVSSFTEYTVVDIAHLVKISPEIPVDIAALLSCSVATGLGAAWKVADVEEGSTVVIFGLGAVGLAVAEGVRLRGAAKIIGVDLNPAKFEIGKRFGITDFVNPALCGEKTISEVIREMTDVGADYSFECIGLASLMEEAFKSTRPGSGKTIVLGMEQKALPISLGSYDLLRGRTVCGTLFGGLKPKLDIPILVDRYLKKELNLEDLITHELSFEEINKAFHLLAEGNSIRCIIWM

SEQ ID NO: 23 - protein, 378 aa, Arabidopsis thaliana
TQGKVITCKAAVAWGAGEPLVMEDVKVDPPQRLEVRIRILFTSICHTDLSAWKGENEAQRAYPRILGHEAAGIVESVGEGVEEMMAGDHVLPIFTGECGDCRVCKRDGANLCERFRVDPMKKVMVTDGKTRFFTSKDNKPIYHFLNTSTFSEYTVIDSACVLKVDPLFPLEKISLLSCGVSTGVGAAWNVADIQPASTVAIFGLGAVGLAVAEGARARGASKIIGIDINPDKFQLGREAGISEFINPKESDKAVHERVMEITEGGVEYSFECAGSIEALREAFLSTNSGVGVTVMLGVHASPQLLPIHPMELFQGRSITASVFGGFKPKTQLPFFITQCLQGLLNLDLFISHQLPFHDINEAMQLLHQGKALRCLLHL

SEQ ID NO: 24 - protein, 378 aa, Arabidopsis thaliana
SSHKPIRCKAAVSRKAGEPLVMEEIMVAPPQPFEVRIRIICTALCHSDVTFWKLQVPPACFPRILGHEAIGVVESVGENVKEVVEGDTVLPTFMPDCGDCVDCKSHKSNLCSKFPFKVSPWMPRYDNSSRFTDLNGETLFHFLNVSSFSEYTVLDVANVVKIDSSIPPSRACLLSCGVSTGVGAAWETAKVEKGSTVVIFGLGSIGLAVAEGARLCGASRIIGVDINPTKFQVGQKFGVTEFVNSMTCEKNRVSEVINEMTDGGADYCFECVGSSSLVQEAYACCRQGWGKTITLGVDKPGSQICLDSFDVLHHGKILMGSLFGGLKAKTHIPILLKRYLSNELELDKFVTHEMKFEEINDAFQLLLEGKCIRCVLWM

SEQ ID NO: 25 - protein, 378 aa, Arabidopsis thaliana
HVSPGGFMRGAVYREPNKPLTIEEFHIPRPKSNEILIKTKACGVCHSDLHVMKGEIPFASPCVIGHEITGEVVEHGPLTDHKIINRFPIGSRVVGAFIMPCGTCSYCAKGHDDLCEDFFAYNRAKGTLYDGETRLFLRHDDSPVYMYSMGGMAEYCVTPAHGLAPLPESLPYSESAILGCAVFTAYGAMAHAAEIRPGDSIAVIGIGGVGSSCLQIARAFGASDIIAVDVQDDKLQKAKTLGATHIVNAAKEDAVERIREITGGMGVDVAVEALGKPQTFMQCTLSVKDGGKAVMIGLSQAGSVGEIDINRLVRRKIKVIGSYGGRARQDLPKVVKLAESGIFNLTNAVSSKYKFEDAGKAFQDLNEGKIVSRGVVEI

SEQ ID NO: 26 - protein, 200 aa, Homo sapiens [sequence omitted]

SEQ ID NO: 27 - protein, 366 aa, Homo sapiens
TTGQVIRCKAAILWKPGAPFSIEEVEVAPPKAKEVRIKVVATGLCGTEMKVLGSKHLDLLYPTILGHEGAGIVESIGEGVSTVKPGDKVITLFLPQCGECTSCLNSEGNFCIQFKQSKTQLMSDGTSRFTCKGKSIYHFGNTSTFCEYTVIKEISVAKIDAVAPLEKVCLISCGFSTGFGAAINTAKVTPGSTCAVFGLGGVGLSVVMGCKAAGAARIIGVDVNKEKFKKAQELGATECLNPQDLKKPIQEVLFDMTDAGIDFCFEAIGNLDVLAAALASCNESYGVCVVVGVLPASVQLKISGQLFFSGRSLKGSVFGGWKSRQHIPKLVADYMAEKLNLDPLITHTLNLDKINEAVELMKTGKW

SEQ ID NO: 28 - protein, 373 aa, Schizosaccharomyces pombe
FEGKTITCKAAVAWGAKEPLSIEDIQVAPPKAHEVRVKVDWSAVCHTDAYTLSGVDPEGAFPIVLGHEGAGIVESIGEGVINVRPGDHVILLYTPECKECKFCRSGKTNLCSKIRETQGRGLMPDGTSRFSCRDKTLLHYMGCSSFSQYTVVADISLVAISHSAPLRSICLLGCGVTTGFGAVTHSAKVESGSTVAVVGCGCVGLAAMQGAVAAGASRIIAIDINADKEVYAKKFGATDFIDSSKVKDLVQYVIDVTDGGVDYAFDCTGNVTVMQQELQFCHKGWGKLCVIGVAAAGKTLDFRPFLVVTGRQVLGSAFGGVKGRSELPNFVDEYMQGHFKVDEYITNEEPLKNINKAFDHMHEGKCIRCVVDM

SEQ ID NO: 29 - protein, 374 aa, Schizosaccharomyces pombe
TAGKIINCKAAVAWQPAAPLSIENVQVFPPRVHEVRIKIVNSGVCHTDAYTLSGKDPEGLFPVILGHEGAGIVESVGPQVTTVQVGDPVIALYTPECKTCKFCKSGKTNLCGRIRTTQGKGLMPDGTSRFSCNGNTLLHFMGCSTFSEYTVVADISVVAIERLAPLDSVCLLGCGITTGYGAATITADIKEGDSVAVFGLGSVGLAVIQGAVKKRAGRIFGIDVNPEKKNWAMSFGATDFINPNDLQSPIQDVLIHETDGGLDWTFDCTGNVHVMRSALEACHKGWGQSIVIGVAAAGQEISTRPFQLVTGRVWRGCAFGGVKGRSQLPDLVKEYLDHKLEIDKYITHRRPLKEINEAFTDMHNGNCIKTVLSI

SEQ ID NO: 30 - protein, 12 aa, Artificial
  Peptide
RRREEEEESAAA

SEQ ID NO: 31 - protein, 375 aa, Artificial
  Consensus
TAGKVITCKAAVAWEAGKPLVIEEIEVAPPKAHEVRIKILATGVCHTDAYVWSGKDPEGLFPVILGHEAAGIVESVGEGVTTVKPGDHVIPLFTPQCGECKFCKSPKTNLCEKFRADNGKGGMPYDGTSRFTCKGKPIYHFMGTSTFSEYTVVDDISVAKIDPSAPLEKVCLLGCGVSTGYGAAWNTAKVEPGSTVAVFGLGGVGLAVAMGAKAAGASRIIGVDINPDKFEKAKEFGATEFINPKDLKKPIQEVIIEMTDGGVDYSFECIGNVSTMRAALESCHKGWGKSVVIGVAAAGQEISTRPFQLVTGRTWKGSAFGGFKSKSDIPKLVKDYMKKKLNLDEFITHELPFEEINKAFDLLHEGKSIRCVLWM

(19) United States
(12) Patent Application Publication
(10) Pub. No.: US 2010/0266581 A1
(43) Pub. Date: Oct. 21, 2010

(54) COMPOSITIONS AND METHODS FOR MODULATING S-NITROSOGLUTHIONE REDUCTASE

(75) Inventors: JONATHAN S. STAMLER, CHAPEL HILL, NC (US); LIMIN LIU, DURHAM, NC (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: DUKE UNIVERSITY, DURHAM, NC (US)

(21) Appl. No.: 12/723,282

(22) Filed: Mar. 12, 2010

Related U.S. Application Data
(63) Continuation of application No. 11/974,367, filed on Oct. 12, 2007, now abandoned.
Application No. 11/974,367 is a division of application No. 10/861,304, filed on Jun. 4, 2004, now abandoned.
(60) Provisional application No. 60/476,055, filed on Jun. 4, 2003.
(60) Provisional application No. 60/545,965, filed on Feb. 18, 2004.
(60) Provisional application No. 60/550,833, filed on Mar. 4, 2004.

(51) Int. Cl. A61K 39/395; A61K 31/155; A61P 25/16; A61P 25/28; A61P 9/10; A61P 35/00; A61P 9/12; A61K 31/7088; A61K 31/713; A61K 31/416; A61K 31/198

(52) U.S. Cl. 424/131.1; 514/637; 424/139.1; 514/44 R; 514/44 A; 424/146.1; 514/406; 514/565

(57) ABSTRACT

Disclosed herein are methods and compositions for modulating the levels and/or activity of S-nitrosoglutathione reductase (GSNOR) in vivo or in vitro. Specifically disclosed are GSNOR deletion constructs, host cells and non-human mammals comprising GSNOR deletions, and methods of screening employing GSNOR deletion mutants. Also specifically disclosed are reagents and procedures for measuring, monitoring, or altering GSNOR levels or activity (as well as nitric oxide and S-nitrosothiol levels) in connection with various medical conditions.

### RELATED APPLICATIONS

This application is a continuation of U.S. Ser. No. 11/974,367, filed Oct. 12, 2007, which is a divisional application of U.S. Ser. No. 10/861,304 filed Jun. 4, 2004, which claims the benefit of U.S. Ser. No. 60/476,055 filed Jun. 4, 2003, U.S. Ser. No. 60/545,965 filed Feb. 18, 2004, and U.S. Ser. No. 60/550,833 filed Mar. 4, 2004. The contents of each of these applications are herein incorporated by reference in their entireties.

### FIELD OF THE INVENTION

This invention relates to nitric oxide (NO) biology. Specifically, this invention relates to the modulation of S-nitrosoglutathione reductase (GSNOR) and nitric oxide bioactivity in the regulation of hemodynamic responses.

### BACKGROUND OF THE INVENTION

Three classes of nitric oxide (NO) synthase (NOS) enzymes play important roles in a wide range of cellular functions and in host defense (Moncada et al., 1991; Nathan and Xie, 1994). The expression, regulation, and activities of these enzymes have been studied extensively through both genetic and pharmacological approaches. The events downstream of NO synthesis are, however, much less well understood. It has been reported that both endogenous and exogenous nitric oxide (NO) react with thiols in proteins such as albumin to form long-lived S-nitrosothiols (SNOs) with vasodilatory activity (Stamler J S, et al., 1992, Proc. Natl. Acad. Sci. USA. 89:444-448). Also reported has been the presence of a circulating pool of S-nitrosoalbumin in plasma whose levels were coupled to NOS activity, such that inhibition of NOS led to a decline in SNO-albumin with concomitant production of low-mass SNOs (Stamler J S, et al., 1992, Proc. Natl. Acad. Sci. USA 89:7674-7677). It was proposed that SNO-albumin provided a reservoir of NO bioactivity that could be utilized in states of NO deficiency, and that vasodilation by SNO-albumin was transduced by the small mass SNOs with which it exists in equilibrium.

Shortly thereafter, it was determined that a key low-mass SNO in biological systems is S-nitrosoglutathione (GSNO; Gaston B, et al., 1993, Proc. Natl. Acad. Sci. USA 1993; 90:10957-10961). In contrast to NO, GSNO retains smooth muscle relaxant activity in the presence of blood hemoglobin, and GSNO acts as a more potent relaxant than SNO-proteins. It was then demonstrated the existence of intraerythrocytic equilibria between NO bound to the thiol of glutathione and reactive thiols (cysβ93) of hemoglobin (Jia L, et al., 1996, Nature 380:221-226), and NO bound to thiols of hemoglobin and membrane-associated band 3 protein (AE1; Pawloski J R, et al., 2001, Nature 409:622-626). The exchange of NO groups between S-nitrosohemoglobin (SNO-Hb) and the red blood cell (RBC) membrane was shown to be governed by O₂ tension (PO₂). Thus, it was found that RBCs dilated blood vessels at low PO₂ (Pawloski J R, et al., 2001, Nature 409:622-626; McMahon T J, et al., 2002, Nat. Med. 8:711-717; Datta B, et al, 2004, Circulation (in press)); and the production of membrane SNO was shown to be required for vasodilation.

In peripheral tissues, experiments have demonstrated that blood flow is determined by variations in hemoglobin O₂ saturation that are coupled to metabolic demand. The mechanism through which the O₂ content of blood evokes this response and the basis for its impairment in many diseases, including heart failure, diabetes, and shock, have been major and longstanding questions in vascular physiology. Previous studies have suggested that the answers reside with hemoglobin's ability to serve as both an O₂ sensor and O₂-responsive transducer of vasodilator activity. It was later determined that albumin and hemoglobin are privileged sites of SNO production. In albumin, both a hydrophobic pocket and bound metals (copper and perhaps heme) can facilitate S-nitrosylation by NO (Foster M W, et al., 2003, Trends Mol. Med. 9:160-168; Rafikova O, et al., 2002, Proc. Natl. Acad. Sci. USA 99:5913-5918). In contrast, hemoglobin (Hb) has several channels through which it can react with NO, nitrite, or GSNO to produce SNO-Hb (Gow A J, et al., 1998, Nature 391:169-173; Gow A J, et al., 1999, Proc. Natl. Acad. Sci. USA. 96:9027-9032; Luchsinger B P, et al., 2003, Proc. Natl. Acad. Sci. USA 100:461-466; Jia L, et al., 1996, Nature 380:221-226; Romeo A A, et al., 2003, J Am. Chem. Soc. 2003; 125:14370-14378).

Additional studies indicated that S-nitrosylation of blood proteins may be catalyzed by superoxide dismutase (SOD), ceruloplasmin, and nitrite. In particular, ceruloplasmin catalyzes the conversion of NO to GSNO (Inoue K, et al., 1999, J. Biol. Chem. 274:27069-27075) and NO in solution or derived from GSNO is targeted by SOD to cysβ93 in hemoglobin rather than heme iron (Gow A J, et al., 1999, Proc. Natl. Acad. Sci. USA 96:9027-9032; Romeo A A, 2003, J. Am. Chem. Soc. 125:14370-14378). A similar mechanism (involving SOD and nitrite) has been postulated to operate in albumin. Numerous laboratories have verified the presence of SNO albumin, GSNO, and SNO-Hb in blood and tissues of both animals and humans. However, the amounts that form, the suitability of various methods for assaying various SNOs, and the physiological roles of these molecules remain in question. It has been proposed that S-nitrosylation of cysteine thiols constitutes a significant route for transduction of NO bioactivity. S-nitrosylation is believed to stabilize and diversify NO-related signals, and act as a ubiquitous regulatory modification for a broad spectrum of proteins (Boehning and Snyder, 2003; Foster et al., 2003; Stamler et al., 2001). Several lines of evidence support this proposition.

First, SNO derivatives of peptides and proteins are present in most tissues and extracellular fluids under basal conditions (Gaston et al., 1993; Gow et al., 2002; Jaffrey et al., 2001; Jia et al., 1996; Kluge et al., 1997; Mannick et al., 1999; Rodriguez et al., 2003; Stamler et al., 1992). Second, there are examples of physiological responses that are uniquely recapitulated by specific SNOs (De Groote et al., 1996; Lipton et al., 2001; Travis et al., 1997). Third, researchers have found that S-nitrosylation/denitroyslation of proteins is dynamically regulated by diverse physiological stimuli across a spectrum of cells types and in vitro systems (Eu et al., 2000; Gaston et al., 1993; Gow et al., 2002; Haendeler et al., 2002; Mannick et al., 1999; Matsumoto et al., 2003; Matsushita et al., 2003; Rizzo and Piston, 2003).

However, investigators lack biochemical or genetic means to distinguish the in vivo activity of SNOs from NO (or other reactive nitrogen species; RNS). Thus, their exact roles and relative importance in various physiological responses remain in question. At basal conditions, NOSs influence arteriolar tone through complex effects on blood vessels, kidneys, and brain (Ortiz and Garvin, 2003; Stamler, 1999; Stoll et al., 2001). In addition, studies from a number of laboratories have pointed toward the role of red blood cells (RBCs), and derived NO bioactivity, in the integrated vascular response that regulates arteriolar resistance (Cirillo et al., 1992; Gonzalez-Alonso et al., 2002; McMahon et al., 2002). NO itself has not been detected in blood or tissues. This has led to the hypothesis that SNOs contribute to vascular homeostasis (Foster et al., 2003; Gow et al., 2002).

Inducible NOS (iNOS) can produce higher output of NO/RNS and thereby disrupt cellular function (Moncada et al., 1991; Nathan and Xie, 1994). This pathophysiological situation, termed nitrosative stress (Hausladen et al., 1996), has been likened to oxidative stress caused by reactive oxygen species (ROS) (Hausladen et al., 1996; Hausladen and Stamler, 1999). Studies of superoxide dismutase, catalase, and peroxidases have provided incontrovertible genetic evidence for an enzymatic defense against ROS. However, the role and mechanism of RNS detoxification in multicellular organisms is unknown. Nonetheless, accumulating evidence points to the existence of a nitrosative stress-response that subserves NO/SNO homeostasis. In particular, iNOS expression coincides with an increase in S-nitrosylated proteins, which rapidly reaches a new steady state level (Eu et al., 2000; Marshall and Stamler, 2002). These data suggest that SNOs are being actively degraded.

Expression of iNOS is strongly induced in septic shock, a complex syndrome that claims over 100,000 human lives per year in the United States alone (Feihl et al., 2001). The role of iNOS in septic and endotoxic shock has been probed extensively in mice. Initial analyses of two independently generated iNOS-deficient (iNOS−/−) mouse lines did not reveal clear differences in mortality when compared with wild-type controls (Laubach et al., 1995; MacMicking et al., 1995). However, more thorough studies of these mice showed that iNOS deficiency actually increased mortality following lipopolysaccharide (LPS) challenge (Laubach et al., 1998; Nicholson et al., 1999). This indicated a protective role for iNOS, which was most apparent in females (Laubach et al., 1998). Consistent with these data, the iNOS inhibitors 1400W and N-(1-iminoethyl)-L-lysine, either have little effect or worsen injury in animal models of endotoxic shock (Feihl et al., 2001; Ou et al., 1997).

Researchers have recently identified a highly conserved S-nitrosoglutathione (GSNO) reductase (GSNOR) (Jensen et al., 1998; Liu et al., 2001). The enzyme is classified as an alcohol dehydrogenase (ADH III; also known as glutathione-dependent formaldehyde dehydrogenase) (Uotila and Koivusalo, 1989), but shows much greater activity toward GSNO than any other substrate (Jensen et al., 1998; Liu et al., 2001). GSNOR appears to be the major GSNO-metabolizing activity in eukaryotes (Liu et al., 2001). Thus, GSNO can accumulate in extracellular fluids where GSNOR activity is low or absent (e.g. airway lining fluid) (Gaston et al., 1993). Conversely, GSNO cannot be detected readily inside cells (Eu et al., 2000; Liu et al., 2001).

Yeast deficient in GSNOR accumulate S-nitrosylated proteins that are not substrates of the enzyme. This indicates that GSNO exists in equilibrium with SNO-proteins (Liu et al., 2001). Such precise control over ambient levels of GSNO and SNO-proteins raises the possibility that GSNO/GSNOR may play roles in both physiological signaling and protection against nitrosative stress. Indeed, GSNO has been implicated in responses ranging from the drive to breathe (Lipton et al., 2001) to regulation of the cystic fibrosis transmembrane regulator (Zaman et al., 2001) and host defense (de Jesus-Berrios et al., 2003). Other studies have found that GSNOR protects yeast cells against nitrosative stress both in vitro (Liu et al., 2001) and in vivo (de Jesus-Berrios et al., 2003).

Currently, there is a great need in the art for diagnostics, prophylaxes, ameliorations, and treatments for medical conditions relating to increased NO synthesis and/or increased NO bioactivity. There is also a need for compositions and methods for blocking the effects of NO, for example, on cell death and cell proliferation, particularly, stem cell proliferation, and vascular homeostasis. In addition, there is a significant need for compositions and methods for preventing, ameliorating, or reversing other NO-associated disorders.

### SUMMARY OF THE INVENTION

The invention relates to methods of alleviating or inhibiting the onset of at least one symptom of a disorder associated with increased levels of nitric oxide bioactivity comprising: administering to a patient (e.g., a female patient) with the disorder a therapeutically effective amount of an agent that increases activity or levels of a S-nitrosoglutathione reductase and/or decreases levels of SNOs (e.g., SNO-Hb). In various aspects of the invention, the disorder is a degenerative disorder (e.g., Parkinson's disease, Alzheimer's disease, amyotrophic lateral sclerosis (ALS)), stroke, systemic infection (e.g., bacteremia, sepsis, neonatal sepsis, septic shock, cardiogenic shock, endotoxic shock, toxic shock syndrome, or systemic inflammatory response syndrome), inflammatory disease (e.g., colitis, inflammatory bowel disease, rheumatoid arthritis, osteoarthritis, psoriatic arthritis, infectious arthritis, ankylosing spondylitis, tendonitis, bursitis, vasculitis, fibromyalgia, polymyalgia rheumatica, temporal arteritis, giant cell arteritis, polyarteritis, HIV-associated rheumatic disease syndromes, systemic lupus, erythematosus, gout, and pseudogout (calcium pyrophosphate dihydrate crystal deposition disease), hypotension (e.g., in connection with anesthesia, dialysis, orthostatic hypotension), proliferative disorders (e.g., cancer or other neoplasms), or another disorder.

In accordance with the invention, this agent may decrease levels of nitric oxide bioactivity or SNOs, or increase nitric oxide/SNO breakdown (e.g., SNO-Hb). In specific aspects, the agent comprises a S-nitrosoglutathione reductase polypeptide (e.g., SEQ ID NO:17-SEQ ID NO:21) or peptide (e.g., peptide encoded by SEQ ID NO:9-SEQ ID NO:14), a S-nitrosoglutathione reductase mimetic (e.g., a peptide, small molecule, or anti-idiotype antibody), a vector for expressing a S-nitrosoglutathione reductase polypeptide (e.g., SEQ ID NO:17-SEQ ID NO:21) or peptide (e.g., peptide encoded by SEQ ID NO:9-SEQ ID NO:14), any fragment, derivative, or modification thereof, or other activator. In certain aspects, the activating agent is co-administered with one or more inhibitor of nitric oxide synthase (e.g., N-[3-(aminomethyl)benzyl]acetamidine (1400W); N6-(1-Iminoethyl)-L-lysine (L-NIL); monomethyl arginine (e.g., for non-specific inhibition); or 7-Nitroindazole (e.g., for inhibition of nNOS in brain tissue), etc.). In a particular embodiment, increased SNOs can be targeted by combination therapy with an S-nitrosoglutathione reductase activator and a nitric oxide synthase inhibitor, or by an S-nitrosoglutathione reductase activator alone.

The invention further relates to methods for alleviating or inhibiting the onset of at least one symptom of a vascular disorder comprising: administering to a patient suffering from the disorder a therapeutically effective amount of an agent that decreases activity or levels of a S-nitrosoglutathione reductase and/or increases levels of SNOs (e.g., SNO-Hb). In various aspects, the vascular disorder is heart disease, heart failure, heart attack, hypertension, atherosclerosis, restenosis, asthma, or impotence. The agent may comprise an antibody (e.g., monoclonal antibody) or antibody fragment that binds to a S-nitrosoglutathione reductase, an antisense or small interfering RNA sequence, a small molecule, or other inhibitor. In certain aspects, the inhibitory agent is co-administered with a phosphodiesterase inhibitor (e.g., rolipram, cilomilast, roflumilast, Viagra® (sildenifil citrate), Cialis® (tadalafil), Levitra® (vardenifil), etc.). In other aspects, the inhibitor is co-administered with a β-agonist, especially for use with heart failure, hypertension, and asthma.

The invention also relates to methods of diagnosing or monitoring a disorder (or treatment of a disorder) associated with increased levels of nitric oxide bioactivity comprising: (a) measuring levels or activity of a S-nitrosoglutathione reductase in a biological sample from a patient (e.g., a female patient); (b) comparing the levels or activity of the S-nitrosoglutathione reductase in the biological sample to levels in a control sample; and (c) determining if the levels or activity of the S-nitrosoglutathione reductase in the biological sample are lower than the levels of the S-nitrosoglutathione reductase in the control sample. In other aspects, the diagnostic or monitoring method comprises (a) measuring levels of SNOs in a biological sample from a patient (e.g., plasma levels); (b) comparing the levels of SNOs in the biological sample to levels in a control sample; and (c) determining if the levels of SNOs in the biological sample are higher than the levels of SNOs in the control sample. Similar diagnostic and monitoring methods are also encompassed for determining increased or deleteriously high levels of S-nitrosoglutathione reductase, or decreased or deleteriously low levels of SNOs.

In various aspects of the invention, the disorder for diagnosis relating to increased levels of nitric oxide bioactivity is a degenerative disease (e.g., Parkinson's disease, Alzheimer's disease, amyotrophic lateral sclerosis), stroke, systemic infection (e.g., bacteremia, sepsis, neonatal sepsis, septic shock, cardiogenic shock, endotoxic shock, toxic shock syndrome, or systemic inflammatory response syndrome), inflammatory diseases (e.g., colitis, inflammatory bowel disease, rheumatoid arthritis, osteoarthritis, psoriatic arthritis, infectious arthritis, ankylo sing spondylitis, tendonitis, bursitis, vasculitis, fibromyalgia, polymyalgia rheumatica, temporal arteritis, giant cell arteritis, polyarteritis, HIV-associated rheumatic disease syndromes, systemic lupus, erythematosus, gout, and pseudogout (calcium pyrophosphate dihydrate crystal deposition disease), hypotension (e.g., associated with anesthesia, dialysis, or orthostatic hypotension), proliferative disease (e.g., cancer, tumor, dysplasia, neoplasm, or precancer lesions) or another disorder. The disorders for diagnosis relating to increased levels of S-nitrosoglutathione reductase and decreased levels of SNOs (e.g., SNO-Hb) include vascular disorder is heart disease, heart failure, heart attack, hypertension, atherosclerosis, restenosis, asthma, or impotence. The diagnostic methods of the invention can employ blood, urine, saliva, or other body fluid or cellular or tissue samples.

In accordance with the invention, the levels of the S-nitrosoglutathione reductase in the biological sample can be determined using an antibody that binds to a S-nitrosoglutathione reductase antigen and/or an antibody that binds to a SNO antigen. In certain embodiments, the antibody is a monoclonal antibody and is, optionally, labeled.

In other embodiments, the levels of the S-nitrosoglutathione reductase in the biological sample are determined using a nucleic acid probe that binds to a S-nitrosoglutathione reductase nucleotide sequence (e.g., SEQ ID NO:7-SEQ ID NO:16 or a complementary sequence). In certain embodiments, the probe is a DNA probe and is, optionally, labeled. Alternatively, the activity of a S-nitrosoglutathione reductase can be determined by known methods. The levels of SNO in a biological sample (e.g., plasma levels) are preferably determined by photolysis-chemiluminescence-based methods. Preferably, stable nitrosothiol standards for, e.g., for SNO-albumin or SNO-Hb measurements, are used in conjunction with such methods.

In addition, the invention relates to transgenic non-human mammals (e.g., mice, rats, etc.) having genomes that comprise a disruption of the endogenous GSNOR gene, wherein the disruption comprises the insertion of a selectable marker sequence, and wherein the disruption results in the mouse exhibiting an increase (e.g., intracellular or extracellular) in nitrosylation compared to a wild-type mouse. In certain aspects, this increase in nitrosylation results in an accumulation of SNOs. The disruption may be a homozygous disruption, for example, that results in a null mutation of the endogenous gene encoding S-nitrosoglutathione reductase, using the neomycin resistance gene as the selectable marker.

The invention further relates to nucleic acids comprising a GSNOR knockout construct comprising a selectable marker sequence flanked by DNA sequences homologous to the endogenous GSNOR gene. Also related are vectors comprising these nucleic acids and host cells and cell lines (e.g., non-human mammal embryonic cell lines) comprising these vectors. Additionally related are methods for identifying an agent for alleviating at least one symptom of a systemic infection or hypotension comprising: (a) administering a test agent to a GSNOR knockout mouse with a systemic infection or hypotension, and (b) determining whether the test agent alleviates a symptom of the systemic infection or hypotension in the knockout mouse. In various aspects, the systemic infection is bacteremia, sepsis, neonatal sepsis, septic shock, endotoxic shock, toxic shock syndrome, or systemic inflammatory response syndrome, while the hypotension is due to anesthesia (e.g., phenobarbitol, ketamine xylazine, or urethane). The symptom may be an increase in nitrosylation, for example, which results in an accumulation of SNOs.

Other embodiments, objects, aspects, features, and advantages of the invention will be apparent from the accompanying description and claims.

### BRIEF DESCRIPTION OF THE DRAWINGS

FIGS. 1A-1F: Targeted Disruption of the GSNOR Gene. FIG. 1A: Strategy for targeted disruption of the GSNOR gene. The structures of the targeting vector, wild-type and disrupted GSNOR alleles are shown. The restriction sites used for construction of the targeting vector and Southern analysis are: B, BamH I; H, Hind III; N, Not I; S, Sac I; X, Xba I. Cassettes PGKneo and PGKtk are the selectable genes neo and tk respectively, under control of the mouse phosphoglycerokinase gene promoter. Double-headed arrows represent expected fragments of wild-type and disrupted GSNOR alleles in Southern analyses with Sac I or Xba I restriction. Neo3′se and GSNOR3′as are the PCR primers used to detect the disrupted allele. FIG. 1B: Southern analysis of genomic DNA from GSNOR-targeted ES clones. The DNA was digested with Sac I and probed with ex2-3, a cDNA probe specific for exons 2-3 of GSNOR. WT, wild-type; KO, disrupted allele. FIG. 1C: Southern analysis of genomic DNA from wild-type (+/+), heterozygous (+/−) and GSNOR (−/−) null mice. DNA was digested with Xba I and hybridized with ex8-9, a probe specific for exons 8-9. FIG. 1D: GSNOR activity in mouse tails. The data include the means (±SD) of 2-4 samples. FIG. 1E: GSNOR activities in various tissues. Protein extracts (500 μg/ml) were incubated with 200 μM NADH and 0 or 150 μM GSNO. Values were obtained from 3 wild-type (filled) or 2 GSNOR^{-/-} (open) mice. FIG. 1F: Body weights of 80-day-old mice (n=18-29) and litter sizes at weaning (n=16-32). Mice from wild-type (open), GSNOR^{-/-} line one (streaked) and line two (filled) were raised on a standard mouse diet in the same animal facility.

FIGS. 2A-2D: Blood Pressure and S-nitrosothiols in Wild-type compared to GSNOR^{-/-} Mice. FIG. 2A: Mean arterial pressure in anesthetized C57BL/6 (WT) and GSNOR^{-/-} (KO) mice. The data include the means±SE of two males and two females in each strain (i.e., n=4 per strain). FIG. 2B: Systolic blood pressure in conscious mice. Data are the means±SE of 8 C57BL/6 (4 males) and 12 GSNOR^{-/-} (4 males) mice. FIG. 2C: Nitrosylation in RBCs from unanesthetized wild-type (open) and GSNOR^{-/-} (filled) mice. SNO-Hb levels in GSNOR^{-/-} mice were determined to be significantly higher than in wild-type mice (P<0.05, n 12), whereas iron-nitrosylHb levels were not different. FIG. 2D: Schematic showing vasodilation by RBC-SNO coupled to hypoxia/metabolic demand by plasma SNOs and vasodilation during NO deficiency states.

FIGS. 3A-3E: Increased Mortality from Endotoxic and Septic Shock in GSNOR^{-/-} Mice. FIG. 3A: Survival of GSNOR^{-/-} mice (filled circles, n=69) was significantly lower than that of wild-type mice (open circles, n=39) following intraperitoneal injection of LPS (P<0.001). FIG. 3B: Survival of mice from GSNOR^{-/-} line one (GSNOR^{-/-}1, upright triangle; n=37) and line two (GSNOR^{-/-}2, inverted triangle; n=32) was similar. Both values were significantly lower than the wild-type mice (open circles, n=39) after LPS (P<0.002 for GSNOR^{-/-}1, P<0.004 for GSNOR^{-/-}2). FIG. 3C: Survival of male GSNOR^{-/-} mice (filled circles, n=31) was not significantly lower than wild-type controls (open circles, n=16) after LPS (P=0.12). FIG. 3D: Survival of both female GSNOR^{-/-}1 (upright triangle; P<0.01, n=19) and female GSNOR^{-/-}2 (inverted triangle; P<0.002, n=19) mice was significantly lower than wild-type controls (open circles, n=23) after LPS. FIG. 3E: Survival of GSNOR^{-/-} female mice (filled, n=9) was significantly lower than that of wild-type controls (open, n=8) following cecal ligation and puncture (CLP; P<0.03).

FIGS. 4A-4E: Abnormal SNO metabolism in GSNOR^{-/-} Mice. FIG. 4A: Liver S-nitrosothiols in wild-type and GSNOR^{-/-} mice after intraperitoneal injection of PBS (48 h) or LPS. Levels of SNO in GSNOR^{-/-} mice were determined to be significantly higher than in wild-type controls at both 24 h (P=0.005) and 48 h (P=0.006) after LPS challenge. FIG. 4B: Serum nitrate in wild-type (open) and GSNOR^{-/-} (filled) mice. Nitrate levels in GSNOR^{-/-} mice were significantly higher (P=0.016) than in wild-type controls at 48 h after LPS. FIG. 4C: Serum nitrite in wild-type (open) and GSNOR^{-/-} (filled) mice. FIG. 4D: Elevated ratios of liver SNO to serum nitrate were significantly higher (P=0.010) at 48 h than 24 h after LPS in GSNOR^{-/-} mice. (Analysis was carried out on mice with significantly elevated nitrate levels (>100 μM)). FIG. 4E: The level of liver SNO was significantly higher (P=0.007) in GSNOR^{-/-} (filled) mice than in wild-type (open) controls at 72 h after CLP.

FIGS. 5A-5H: Serum Markers of Tissue Injury. Serum was collected 48 h following control PBS injection and 24 h or 48 h following LPS injection. Data (mean±SE) were obtained from 4-12 wild-type (open) or GSNOR^{-/-} (filled) mice. Significant pair-wise differences are indicated by an asterisk (p<0.015). Markers assayed were: (FIG. 5A) alanine aminotransferase (ALT); (FIG. 5B) aspartate aminotransferase (AST); (FIG. 5C) creatinine; (FIG. 5D) urea nitrogen (BUN); (FIG. 5E) creatine phosphokinase (CPK); (FIG. 5F) amylase; (FIG. 5G) lipase. FIG. 5H: Correlation between ALT (R²=0.85, p<0.01) or AST (R²=0.94, p<0.01) and liver SNO in six GSNOR^{-/-} mice (48 h after LPS).

FIGS. 6A-6H: Histopathology of LPS-Challenged Mice. Shown are sections of liver (FIGS. 6A-6B), thymus (FIGS. 6C-6D), spleen (FIGS. 6E-6F), and mesenteric (pancreatic) lymph node (FIGS. 6G-6H) of wild-type (FIGS. 6A, 6C, 6E and 6G) and GSNOR^{-/-} (FIGS. 6B, 6D, 6F and 6H) mice 48 hours after LPS. All the micrographs are of the same magnification, and the scale bar in (FIG. 6A) is 20 μm. N, necrotic hepatocyte; T, tingible body macrophage with phagocytosed apoptotic cells. Each micrograph is representative of three animals.

FIGS. 7A-7D: iNOS Inhibition Prevents SNO Elevation, Reduces Liver Injury, and Improves Survival of LPS-Challenged GSNOR^{-/-} Mice. FIGS. 7A-7C: Serum levels of nitrate (FIG. 7A; n=7), liver S-nitrosothiol (FIG. 7B; n=4) and serum ALT (FIG. 7C; n=5) in GSNOR^{-/-} mice that were given 1400W 6 h following LPS injection (filled columns). Open columns represent the values obtained in the absence of 1400W and are reproduced from FIGS. 4A, 4B and 5A. FIG. 7D: Survival of LPS-challenged GSNOR^{-/-} mice that received either 1400W (n=12; squares) or PBS (n=6; diamonds) 6 h following LPS injection.

FIG. 8 shows the amount of airway resistance treated with increasing amounts of methylcholine (MCh) wild-type mice and GSNOR^{-/-} mice treated with ovalbumin (OVA) and PBS.

FIG. 9 shows the level of IgE in both wild-type and GSNOR^{-/-} mice after treatment with OVA or PBS.

FIG. 10 shows the level of BALF IL-13 in OVA treated GSNOR^{-/-} and wild-type mice.

FIGS. 11A-11B. Results from GRK studies. FIG. 11A. Representative gel from experiments examining the effect of cysNO (500, 50, and 5 μM) on isoproterenol (10 μM) stimulated GRK2 mediated receptor phosphorylation using purified β₂-AR reconstituted in synthetic vesicles and purified GRK2. FIG. 11B: Representative gel from experiments examining the effect of cysNO (5, 50, and 500 μM) light stimulated GRK2 mediated phosphorylation of rhodopsin using purified bovine rod outer segments and purified GRK2.

FIGS. 12A-12B: Effect of cysNO (A: 500, 50, and 5 μM) and GSNO (B: 500, 50, and 5 μM) on purified GRK2 mediated in vitro phosphorylation of a soluble peptide substrate (RRREEEEESAAA; SEQ ID NO:30) (n=2; *P<0.05).

FIGS. 13A-13C: Results of Cardiac Studies. FIG. 13A: Heart weight to body weight ratio (hw:bw, mg:g)(n=10); FIG. 13B: Cardiac β-AR density (BMax, fmol/mg protein (n=5); FIG. 13C: Cardiac PARK protein expression levels (n=4), in mice following mini-osmotic pump implantation and treatment for 7 days with either PBS, isoproterenol (ISO) (30 mg/kg/day), GSNO (10 mg/kg/day) or a combination of ISO and GSNO. All data expressed as mean (+/−SEM) (*P<0.05 versus PBS treated mice, ⁺P<0.05 versus ISO treated mice, unpaired t test).

FIGS. 14A-14B: Amino Acid Sequence Alignment for Human GSNOR and Homologous or Orthologous Sequences. Amino acid sequence information (SEQ ID NO:21-SEQ ID NO:29, consecutively) and sequence alignment was obtained from NCBI Conserved Domain Database CD: KOG0022.1, KOG0022. In the alignment, Accession No. 1MC5_A (SEQ ID NO: 21) corresponds to human GSNOR; GenBank No. 113389 (SEQ ID NO: 27) corresponds to human alcohol dehydrogenase 6; GenBank No. 174441816 (SEQ ID NO:26) corresponds to a sequence similar to human class IV alcohol dehydrogenase; GenBank No. 13432155 (SEQ ID NO: 28) corresponds to glutathione-dependent formaldehyde dehydrogenase 1 from Schizosaccharomyces pombe; GenBank No. 13431519 (SEQ ID NO: 29) corresponds to glutathione-dependent formaldehyde dehydrogenase 2 from Schizosaccharomyces pombe; GenBank No. 30697873 (SEQ ID NO: 25) corresponds to oxidoreductase from Arabidopsis thaliana; GenBank No. 15238330 (SEQ ID NO: 24) corresponds to an alcohol dehydrogenase sequence from Arabidopsis thaliana; GenBank No. 15217715 (SEQ ID NO: 23) corresponds to an alcohol dehydrogenase sequence from Arabidopsis thaliana; GenBank No. 15219884 (SEQ ID NO: 22) corresponds to an alcohol dehydrogenase sequence from Arabidopsis thaliana. Conserved domains are shown in bold. Positions with conservative substitutions are shown in bold, with italics.

### DETAILED DESCRIPTION OF INVENTION

### Definitions

As used herein, “protein” is used synonymously with “polypeptide”. A “purified” polypeptide, protein, or peptide is substantially free of cellular material or other contaminating proteins from the cell, tissue, or cell-free source from which the amino acid sequence is obtained, or substantially free from chemical precursors or other chemicals when chemically synthesized.

The language “substantially free of cellular material” includes preparations of polypeptides or peptides that are separated from cellular components of the cells from which the amino acid sequences are isolated or recombinantly produced. In one embodiment, the language “substantially free of cellular material” includes preparations of a polypeptide or peptide having less than about 30% (by dry weight) of other proteins (also referred to herein as a “contaminating protein”), more preferably less than about 20% of contaminating protein, still more preferably less than about 10% of contaminating protein, and most preferably less than about 5% contaminating protein. When a polypeptide or peptide is recombinantly produced, it is also preferably substantially free of culture medium, e.g., culture medium represents less than about 20%, more preferably less than about 10%, and most preferably less than about 5% of the volume of the preparation.

The term “antibody” as used herein refers to immunoglobulin molecules and immunologically active portions of immunoglobulin molecules, e.g., molecules that contain an antigen binding site that specifically binds (immunoreacts with) an antigen, such as a polypeptide or peptide. Such antibodies include, e.g., polyclonal, monoclonal, chimeric, single chain, Fab and F(ab′)2 fragments, and an Fab expression library. In specific embodiments, antibodies are generated against human polypeptides, e.g., one or more GSNORs.

The term “monoclonal antibody” or “monoclonal antibody composition”, as used herein, refers to a population of antibody molecules that contain only one species of an antigen binding site capable of immunoreacting with a particular epitope of a polypeptide or peptide. A monoclonal antibody composition thus typically displays a single binding affinity for a particular amino acid sequence with which it immunoreacts.

As used herein, “modulate” is meant to refer to an increase or decrease the levels of a polypeptide, or to increase or decrease the stability or activity of a polypeptide. Thus, an agent can be tested for its ability to activate a polypeptide, or to promote the synthesis or stability of a polypeptide.

As used herein, the term “derivative” or “derived” refers to a chemical substance that is related structurally to another substance and theoretically derivable from it, e.g., a truncated protein or peptide.

As used herein, the term “region” or “domain”, as in protein region or domain, refers to a number of amino acids in a defined area of a parent protein.

As used herein, the term “physiological levels” refer to a characteristic of or appropriate to an organism's healthy or normal functioning. As used herein, the term “physiologically compatible” refers to a solution or substance, for example media, that can be utilized to mimic an organism's healthy or normal environment. For in vivo use, the physiological compatible solution may include pharmaceutically acceptable carriers, excipients, adjuvants, stabilizers, and vehicles.

As utilized herein, the term “pharmaceutically acceptable” means approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

The terms “cell culture medium” and “culture medium” refer to a nutrient solution used for growing cells that typically provides at least one component from one or more of the following categories: 1) an energy source, usually in the form of a carbohydrate such as glucose; 2) all essential amino acids, and usually the basic set of twenty amino acids plus cysteine; 3) vitamins and/or other organic compounds required at low concentrations; 4) free fatty acids; and 5) trace elements, where trace elements are defined as inorganic compounds or naturally-occurring elements that are typically required at very low concentrations, usually in the micromolar range.

For mammalian cells, the cell culture medium is generally “serum free” when the medium is essentially free of serum from any mammalian source (e.g. fetal bovine serum (FBS)). By “essentially free” is meant that the cell culture medium comprises between about 0-5% serum, preferably between about 0-1% serum, and most preferably between about 0-0.1% serum. Advantageously, serum-free “defined” medium can be used, wherein the identity and concentration of each of the components in the medium is known (i.e., an undefined component such as bovine pituitary extract (BPE) is not present in the culture medium).

As defined herein “specific binding” refers to the ability of a protein, peptide, or antigen to interact with an antibody or each other.

As used here, the term “nitric oxide” encompasses uncharged nitric oxide (NO) and charged nitric oxide species, particularly including nitrosonium ion (NO⁺) and nitroxyl ion (NO⁻). The reactive form of nitric oxide can be provided by gaseous nitric oxide. Compounds having the structure X—NO_{y} wherein X is a nitric oxide releasing, delivering or transferring moiety, including any and all such compounds which provide nitric oxide to its intended site of action in a form active for their intended purpose, and Y is 1 or 2.

As used herein, the term “bioactivity” indicates an effect on one or more cellular or extracellular process (e.g., via binding, signaling, etc.) which can impact physiological or pathophysiological processes.

The term “treating” in its various grammatical forms in relation to the present invention includes preventing, curing, reversing, attenuating, alleviating, minimizing, suppressing or halting at least one deleterious symptom or effect of a disease (disorder) state, disease progression, disease causative agent (e.g., bacteria or viruses), or other abnormal condition.

As used herein, “gene therapy” includes both conventional gene therapy where a lasting effect is achieved by a single treatment, and the administration of gene therapeutic agents, which involves the one time or repeated administration of a therapeutically effective DNA or mRNA.

The phrase “SEQ ID NO:7-SEQ ID NO:16,” and the like, is used herein for convenience, and may refer to each SEQ ID NO individually or more than one SEQ ID NO in accordance with the methods of the invention.

A “biological sample” for diagnostic testing includes, but is not limited to, samples of blood (e.g., serum, plasma, or whole blood), urine, saliva, sweat, breast milk, vaginal secretions, semen, hair follicles, skin, teeth, bones, nails, or other secretions, body fluids, tissues, or cells.

The headings for the subsequent sections are provided for organizational purposes only. They are not to be considered limiting.

### Polypeptides

The invention encompasses GSNOR polypeptides (e.g., SEQ ID NO:17-SEQ ID NO:21), peptides (e.g., peptides encoded by SEQ ID NO:9-SEQ ID NO:14), and fragments, variants, modifications, and derivatives thereof. Such polypeptides or peptides can be made using techniques known in the art. For example, one or more of the polypeptides or peptides can be chemically synthesized using art-recognized methods. For example, a peptide synthesizer can be used. See, e.g., Peptide Chemistry, A Practical Textbook, Bodasnsky, Ed. Springer-Verlag, 1988; Merrifield, Science 232:241-247 (1986); Barany, et al, Intl. J. Peptide Protein Res. 30:705-739 (1987); Kent, Ann. Rev. Biochem. 57:957-989 (1988), and Kaiser, et al, Science 243:187-198 (1989).

Alternatively, GSNOR polypeptides or peptides can be made by expressing one or more amino acid sequences from a nucleic acid sequence. Any known nucleic acids that express the polypeptides or peptides (e.g., human or chimerics) can be used, as can vectors and cells expressing these polypeptides or peptides. Sequences of human ORFs and polypeptides are publicly available, e.g. in GenBank and other databases. If desired, the polypeptides or peptides can be recovered and isolated.

Recombinant cells expressing the polypeptide, or a fragment or derivative thereof, may be obtained using methods known in the art, and individual gene products or fragments may be isolated and analyzed (e.g., as described in Sambrook et al., eds., MOLECULAR CLONING: A LABORATORY MANUAL, 2nd Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993).

Assays may be used based upon the physical and/or functional properties of the polypeptides or peptides. The assays can include, e.g., radioactive labeling of one or more of the polypeptides, followed by analysis by gel electrophoresis and immunoassay. Polypeptides and peptides may be isolated and purified by standard methods known in the art either from natural sources or recombinant host cells expressing the proteins/peptides. These methods can include, for example, column chromatography (e.g., ion exchange, affinity, gel exclusion, reverse-phase, high pressure, fast protein liquid, etc.), differential centrifugation, differential solubility, or similar methods used for the purification of proteins.

In certain aspects of the invention, particular domains of the GSNOR polypeptides can be used. Highly conserved domains in human GSNOR include amino acids 17-172 and amino acids 193-241, as well as amino acids 64-80 and amino acids 215-228 (FIGS. 14A-14B). Less conserved domains in human GSNOR include amino acids 1-16 and amino acids 172-193, as well as amino acids 242-374 (FIGS. 18A-18B). In other aspects, conservative variants of these polypeptides or polypeptide domains can be used.

Nucleic acids encoding one or more GSNOR polypeptide or peptide, as well as vectors and cells comprising these nucleic acids, are within the scope of the present invention. Host-vector systems that can be used to express the polypeptides or peptides include, e.g.: (i) mammalian cell systems which are infected with vaccinia virus, adenovirus; (ii) insect cell systems infected with baculovirus; (iii) yeast containing yeast vectors; or (iv) bacteria transformed with bacteriophage, DNA, plasmid DNA, or cosmid DNA. Depending upon the host-vector system utilized, any one of a number of suitable transcription and translation elements may be used.

The expression of the specific polypeptides or peptides may be controlled by any promoter/enhancer known in the art including, e.g.: (i) the SV40 early promoter (see e.g., Bernoist & Chambon, Nature 290:304-310 (1981)); (ii) the promoter contained within the 3′-terminus long terminal repeat of Rous Sarcoma Virus (see e.g., Yamamoto, et al., Cell 22:787-797 (1980)); (iii) the Herpesvirus thymidine kinase promoter (see e.g., Wagner, et al., Proc. Natl. Acad. Sci. USA 78:1441-1445 (1981)); (iv) the regulatory sequences of the metallothionein gene (see e.g., Brinster, et al., Nature 296:39-42 (1982)); (v) prokaryotic expression vectors such as the β-lactamase promoter (see e.g., Villa-Kamaroff, et al., Proc. Natl. Acad. Sci. USA 75:3727-3731 (1978)); (vi) the tac promoter (see e.g., DeBoer, et al., Proc. Natl. Acad. Sci. USA 80:21-25 (1983)).

Plant promoter/enhancer sequences within plant expression vectors may also be utilized including, e.g.,: (i) the nopaline synthetase promoter (see e.g., Herrar-Estrella, et al., Nature 303:209-213 (1984)); (ii) the cauliflower mosaic virus 35S RNA promoter (see e.g., Garder, et al., Nucl. Acids Res. 9:2871 (1981)) and (iii) the promoter of the photosynthetic enzyme ribulose bisphosphate carboxylase (see e.g., Herrera-Estrella, et al., Nature 310:115-120 (1984)).

Promoter/enhancer elements from yeast and other fungi (e.g., the Gal4 promoter, the alcohol dehydrogenase promoter, the phosphoglycerol kinase promoter, the alkaline phosphatase promoter), as well as the following animal transcriptional control regions, which possess tissue specificity and have been used in transgenic animals, may be utilized in the production of proteins of the present invention.

Other animal transcriptional control sequences derived from animals include, e.g.,: (i) the insulin gene control region active within pancreatic β-cells (see e.g., Hanahan, et al., Nature 315:115-122 (1985)); (ii) the immunoglobulin gene control region active within lymphoid cells (see e.g., Grosschedl, et al., Cell 38:647-658 (1984)); (iii) the albumin gene control region active within liver (see e.g., Pinckert, et al., Genes and Devel. 1:268-276 (1987)); (iv) the myelin basic protein gene control region active within brain oligodendrocyte cells (see e.g., Readhead, et al., Cell 48:703-712 (1987)); and (v) the gonadotrophin-releasing hormone gene control region active within the hypothalamus (see e.g., Mason, et al., Science 234:1372-1378 (1986)).

The vector may include a promoter operably-linked to nucleic acid sequences which encode a GSNOR polypeptide or peptide, one or more origins of replication, and optionally, one or more selectable markers (e.g., an antibiotic resistance gene). A host cell strain may be selected which modulates the expression of polypeptide or peptide sequences, or modifies/processes the expressed sequences in a desired manner. Moreover, different host cells possess characteristic and specific mechanisms for the translational and post-translational processing and modification (e.g., glycosylation, phosphorylation, and the like) of expressed polypeptides or peptides. Appropriate cell lines or host systems may thus be chosen to ensure the desired modification and processing of the polypeptide or peptide is achieved. For example, protein expression within a bacterial system can be used to produce an unglycosylated core protein; whereas expression within mammalian cells can be used to obtain native glycosylation of a heterologous protein.

Prokaryotic host cells include gram-negative or gram-positive organisms. Suitable prokaryotic host cells for transformation include, for example, E. coli, Bacillus subtilis, Salmonella typhimurium, and various other species within the genera Pseudomonas, Streptomyces, and Staphylococcus. Alternatively, the polypeptides or peptides may be expressed in yeast host cells, preferably from the Saccharomyces genus (e.g., S. cerevisiae). Other genera of yeast, such as Schizosaccharomyces, Pichia, or Kluyveromyces, may also be employed.

Mammalian or insect host cell culture systems may be used to express recombinant polypeptides or peptides. Baculovirus systems for production of heterologous proteins in insect cells are well known (see, e.g., Luckow and Summers, Bio/Technology 6:47 (1988)). Established cell lines of mammalian origin also may be employed. Examples of suitable mammalian host cell lines include, but are not limited to, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70; McMahan et al. EMBO J. 10: 2821, 1991).

### Nucleic Acids

The invention encompasses GSNOR nucleic acids (e.g., SEQ ID NO:7-SEQ ID NO:16 and sequences encoding SEQ ID NO:17-SEQ ID NO:21), and fragments, variants, derivatives, and complementary sequences thereof. Sequences of human GSNOR genes and coding sequences are publicly available, e.g. in GenBank and other databases. GSNOR nucleic acids can be used, for example, for hybridization probes, in chromosome and gene mapping and in the generation of anti-sense RNA and DNA, small interfering RNAs, and gene therapy vectors (see, e.g., U.S. Published Application 2004/0023323). Such nucleic acids are also useful for the preparation of GSNOR polypeptides and by the recombinant techniques previously described.

The full-length sequence of the GSNOR gene, or portions thereof, may be used as hybridization probes to detect (or determine levels of) GSNOR expression, or to detect variants of GSNOR (e.g., SNPs), or GSNOR nucleic acids from other species. Optionally, the length of the probes will be about 20 to about 50 bases. The hybridization probes may be derived from at least partially novel regions of the full length native nucleotide sequence wherein those regions may be determined without undue experimentation, or from genomic sequences including promoters, enhancer elements, and introns of native sequence of GSNOR.

As one example, a screening method may comprise isolating the coding region of the GSNOR gene using the known DNA sequence to synthesize a selected probe of about 40 bases. Hybridization probes may be labeled by a variety of labels, including radionucleotides such as ³²P or ³⁵S, or enzymatic labels such as alkaline phosphatase, coupled to the probe (e.g., via avidin/biotin coupling systems). Any GSNOR EST sequences may be employed as probes, using the methods disclosed herein.

Other useful GSNOR nucleic acids include antisense or sense oligonucleotides comprising a singe-stranded nucleic acid sequence (either RNA or DNA) capable of binding to target GSNOR mRNA or GSNOR DNA sequences. Binding of oligonucleotides to target nucleic acid sequences can be used to form duplexes that block transcription or translation of the target sequence. Oligonucleotide binding may cause enhanced degradation of the duplexes, premature termination of transcription or translation, or another inhibitory effect. Thus, the oligonucleotides may be used to decrease expression of a GSNOR polypeptide. For example, an antisense RNA or DNA molecule can directly block the translation of mRNA by hybridizing to targeted mRNA and preventing protein translation, or by hybridizing to targeted DNA to form triple-helixes.

Such oligonucleotides, according to the present invention, comprise a fragment of GSNOR DNA, e.g., a fragment of the coding sequence or complementary sequence thereto. Such a fragment generally comprises at least about 14 nucleotides, preferably from about 14 to 30 nucleotides. The design of such oligonucleotides based upon a cDNA sequence has been previously described (see, e.g., Stein and Cohen Cancer Res. 48:2659, 1988; van der Krol et al. BioTechniques 6:958, 1988). These short antisense oligonucleotides can be imported into cells where they act as inhibitors, even where there is low intracellular concentrations caused by their restricted uptake by the cell membrane (Zamecnik et al., Proc. Natl. Acad. Sci. USA 83:4143-4146 (1986)).

For use with the methods of the invention, oligonucleotides can include modified sugar-phosphodiester backbones or other sugar linkages (see, e.g., WO 91/06629). Such oligonucleotides with sugar linkages exhibit increased stability in vivo (i.e., are capable of resisting enzymatic degradation) but retain sequence specificity to be able to bind to target nucleotide sequences. In other aspects, oligonucleotides can be covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the oligonucleotide for the target nucleotide sequence.

Oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see, e.g., WO 90/13641).

Oligonucleotides also may be introduced into a cell containing the target nucleotide sequence by formation of a conjugate with a ligand binding molecule (e.g., as in WO 91/04753). Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its cognate ligand(s), or block entry of the oligonucleotide or its conjugated version into the cell. Alternatively, an oligonucleotide may be introduced into a cell containing the target nucleic acid sequence by formation of an oligonucleotide-lipid complex (see, e.g., WO 90/10448). The oligonucleotide-lipid complex is preferably dissociated within the cell by an endogenous lipase.

Antisense (or sense) RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more. The oligonucleotides can be modified to enhance their uptake, e.g. by substituting their negatively charged phosphodiester groups by uncharged groups.

An oligonucleotide can be designed to be complementary to a region of a transcript or the gene involved in transcription (see Lee et al., Nucl. Acids Res., 6:3073 (1979); Cooney et al., Science, 241:456 (1988); Dervan et al., Science, 251:1360 (1991); Okano, Neurochem., 56:560 (1991); Oligodeoxynucleotides as Antisense Inhibitors of Gene Expression CRC Press: Boca Raton, Fla., 1988). To target a transcript, the 5′ coding portion of the GSNOR polynucleotide sequence can be used to design an antisense oligonucleotide of from about 10 to 40 base pairs in length. To target the gene, oligodeoxyribonucleotides derived from the translation-initiation site, e.g., between about −10 and +10 positions of the target gene nucleotide sequence can be used. Nucleic acid molecules for triple-helix formation can be using via Hoogsteen base-pairing rules, which generally require sizeable stretches of purines or pyrimidines on one strand of a duplex. See, e.g., WO 97/33551.

Other useful nucleic acids include ribozymes, which are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques (see, e.g., Rossi, Current Biology, 4:469-471 (1994), and WO 97/33551).

In another approach, interfering RNAs (iRNAs; Tijsterman, M., et al., 2002, Annu. Rev. Genet. 36, 489-519; Tabara, H., et al., 2002, Cell 109, 861-871) can be used to “knock-down” GSNOR expression. iRNAs are double stranded molecules that are cleaved in the cell by an RNase III like enzyme into small (21 to 23 nucleotides) interfering RNAs (siRNAs; Bernstein, E., et al., 2001, Nature 409, 363-366; Ketting, R. F., et al., 2001, Genes Dev. 15, 2654-2659; Knight, S. W. and Bass, B. L., 2001, Science 293, 2269-2271; Zamore, P. D., et al., 2000, Cell 101, 25-33). siRNAs associate with a large multiprotein complex, the RISC, which unwinds the siRNA to help target the appropriate mRNA (Martinez, J., et al., 2002, Cell 110, 563-574). The siRNA-mRNA hybrid is then cleaved, the siRNA is released, and the mRNA is degraded by endo- and exonucleases (reviewed in Dillin, 2003, Proc. Natl. Acad. Sci. USA, 100: 6289-6291).

In mammalian cells, siRNAs can be added directly to the cells to lead to a specific depletion of the targeted mRNA and consequently the encoded protein product. Such siRNAs can be made synthetically or by use of expression vectors. siRNAs can be designed using known methods (Elbashir S M, et al., 2001, Nature 411: 494-498) and algorithms (see, e.g., Cenix BioScience, Dresden, Germany). In addition, siRNAs and siRNA expression vectors can be obtained from commercial sources (see, e.g., Ambion, Inc., Austin, Tex.; QIAGEN, Inc., Valencia, Calif.; Promega, Madison Wis.; InvivoGen, San Diego, Calif.). Advantageously, siRNAs may be useful for specifically targeting a GSNOR transcript, and leaving related sequences unaffected.

Nucleic acids which encode GSNOR or its modified forms can also be used to generate transgenic animals or cell lines, or knock out animals or cell lines. Transgenics and knock outs are useful in the development and screening of therapeutically useful reagents, as described below. A transgenic animal (e.g., a mouse or rat) is an animal having cells that contain a transgene, which was introduced into the animal or an ancestor of the animal at a prenatal, for example, an embryonic stage. Methods for generating transgenic animals, particularly animals such as mice or rats, are now conventional in the art and are described, for example, in U.S. Pat. Nos. 4,736,866 and 4,870,009.

In one approach, particular cells can be targeted for GSNOR transgene incorporation with tissue-specific enhancers. Animals that include a copy of a transgene encoding GSNOR introduced into the germ line of the animal at an embryonic stage can be used to examine the effect of increased expression of GSNOR. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, as demonstrated herein, non-human homologs (i.e., orthologs) of GSNOR can be used to construct a knock out animal which has a defective or altered gene encoding GSNOR. Knock outs can be produced by homologous recombination between the endogenous gene encoding GSNOR and altered genomic DNA encoding GSNOR introduced into an embryonic stem cell of the animal. For example, cDNA encoding GSNOR can be used to clone genomic DNA encoding GSNOR in accordance with established techniques. A portion of the genomic DNA encoding GSNOR can be deleted or replaced with another gene, such as a gene encoding a selectable marker which can be used to monitor integration.

In one approach, a vector includes several kilobases of unaltered flanking DNA both at the 5′ and 3′ ends (see, e.g., Thomas and Capecchi, Cell, 51:503 (1987)). The vector is introduced into an embryonic stem cell line (e.g., by electroporation) and cells in which the introduced DNA has homologously recombined with the endogenous DNA are selected (see e.g., Li et al., Cell, 69:915 (1992)). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson, ed. (IRL, Oxford, 1987), pp. 113-152).

A chimeric embryo can be implanted into a suitable pseudopregnant female foster animal and the embryo brought to term to create a knock out animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions (e.g., LPS challenge) and for their development of pathological conditions (e.g., hypotension) due to absence of the GSNOR polypeptide.

Nucleic acids encoding GSNOR polypeptides or peptides may also be used in gene therapy. In particular, a GSNOR coding sequence can be introduced into cells to produce a therapeutically effective GSNOR product, for example to replace a defective gene or to increase gene expression. There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc.

In one preferred method, gene transfer is performed in vivo by transfection with viral (typically retroviral) vectors and viral coat protein-liposome mediated transfection (Dzau et al., Trends in Biotechnology 11, 205-210 (1993)). In some situations, it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis has been previously described (see, e.g., Wu et al., J. Biol. Chem. 262, 4429-4432 (1987); and Wagner et al., Proc. Natl. Acad. Sci. USA 87, 3410-3414 (1990)). For review of gene marking and gene therapy protocols see Anderson et al., Science 256, 808-813 (1992).

### Antibodies

The invention further encompasses antibodies and antibody fragments (such as Fab or F(ab′)2 fragments) that bind specifically to a GSNOR polypeptide (e.g., SEQ ID NO:17-SEQ ID NO:21) peptide (e.g., peptide encoded by SEQ ID NO:9-SEQ ID NO:14), or fragment thereof. An antibody that “specifically binds” is one that recognizes and binds to a particular GSNOR amino acid sequence, but which does not substantially recognize or bind to other molecules in a biological sample. In one approach, a purified polypeptide or a portion, variant, or fragment thereof, can be used as an immunogen to generate antibodies that specifically bind the amino acid sequence using standard techniques for polyclonal and monoclonal antibody preparation.

A full-length polypeptide can be used, if desired. Alternatively, antigenic fragments of polypeptides can be used as immunogens. In some embodiments, the antigenic fragment includes at least 6, 8, 10, 15, 20, or 30 or more amino acid residues of a polypeptide. In one embodiment, epitopes include specific domains of the polypeptide, or are located on the surface of the polypeptide, e.g., hydrophilic regions. If desired, peptides containing antigenic regions can be selected using hydropathy plots showing regions of hydrophilicity and hydrophobicity. These plots may be generated by any method well known in the art, including, for example, the Kyte Doolittle or the Hopp Woods methods, either with or without Fourier transformation. See, e.g., Hopp and Woods, Proc. Nat. Acad. Sci. USA 78:3824-3828 (1981); Kyte and Doolittle, J. Mol. Biol. 157:105-142 (1982).

Various procedures known within the art may be used for the production of polyclonal or monoclonal antibodies. For example, for the production of polyclonal antibodies, various suitable host animals (e.g., rabbit, goat, mouse or other mammal) may be immunized by injection with the native polypeptide, or a variant thereof, or a fragment or derivative of the foregoing. An appropriate immunogenic preparation can contain, for example, a recombinantly expressed polypeptide. Alternatively, the immunogenic polypeptides or peptides may be chemically synthesized, as previously discussed.

The immunogenic preparation can further include an adjuvant. Various adjuvants used to increase the immunological response include, e.g., Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against a polypeptide or peptide can be isolated from the mammal (e.g., from the blood) and further purified by well known techniques, such as protein A chromatography to obtain the IgG fraction.

Any technique may be used to prepare monoclonal antibodies directed towards a particular polypeptide or peptide. For example, continuous cell line cultures may be utilized as in, e.g., hybridoma techniques (see Kohler & Milstein, Nature 256:495-497 (1975)); trioma techniques; human B cell hybridoma techniques (see Kozbor, et al., Immunol Today 4:72 (1983)); and EBV hybridoma techniques to produce human monoclonal antibodies (see, Cole, et al., In: Monoclonal Antibodies and Cancer Therapy, Alan R. Liss, Inc., (1985) pp. 77-96). If desired, human monoclonal antibodies may be prepared by using human hybridomas (see Cote, et al., Proc. Natl. Acad. Sci. USA 80:2026-2030 (1983)) or by transforming human B cells with Epstein Barr Virus in vitro (see Cole, et al., In: Monoclonal Antibodies and Cancer Therapy, supra).

Methods can be adapted for the construction of Fab expression libraries (see e.g., Huse, et al., Science 246:1275-1281 (1989)) to allow rapid and effective identification of monoclonal Fab fragments with the desired specificity for the desired protein or derivatives, fragments, analogs or homologs thereof. Non-human antibodies can be “humanized” by techniques well known in the art (see e.g., U.S. Pat. No. 5,225,539). Antibody fragments that contain the idiotypes to a polypeptide or peptide may be produced by techniques known in the art including, e.g.: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent; and (iv) Fᵥ fragments.

Chimeric and humanized monoclonal antibodies against the polypeptides or peptides described herein are also within the scope of the invention. Such antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al., Science 240:1041-1043 (1988); Liu et al., Proc. Nat. Acad. Sci. USA 84:3439-3443 (1987); Liu et al., J. Immunol. 139:3521-3526 (1987); Sun et al., Proc. Nat. Acad. Sci. USA 84:214-218 (1987); Nishimura et al., Cancer Res. 47:999-1005 (1987); Wood et al., Nature 314:446-449 (1985); Shaw et al., J. Natl. Cancer Inst. 80:1553-1559 (1988); Morrison, Science 229:1202-1207 (1985); Oi et al., BioTechniques 4:214 (1986); U.S. Pat. No. 5,225,539; Jones et al., Nature 321:552-525 (1986); Verhoeyan et al., Science 239:1534 (1988); and Beidler et al., J. Immunol. 141:4053-4060 (1988).

Methods for the screening of antibodies that possess the desired specificity include, e.g., enzyme-linked immunosorbent assay (ELISA) and other immunologically-mediated techniques known within the art. For example, selection of antibodies that are specific to a particular domain of a polypeptide can be facilitated by generation of hybridomas that bind to the polypeptide or fragment thereof, possessing such a domain.

In certain embodiments of the invention, antibodies specific for the GSNOR polypeptides or peptides described herein may be used in various methods, such as detection or inhibition of amino acid sequences, and identification of agents which inhibit these sequences. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include GFP, luciferase, luciferin, and aequorin, and examples of suitable radioactive material include ¹²⁵I, ¹³¹I, ³⁵S or ³H.

Polypeptide-specific or peptide-specific antibodies can also be used to isolate amino acid sequences using standard techniques, such as affinity chromatography or immunoprecipitation. Thus, the antibodies disclosed herein can facilitate the purification of specific polypeptides or peptides from cells, as well as recombinantly produced polypeptides or peptides expressed in host cells.

### Diagnostic Methods and Kits

Methods of determining expression and activity levels of a GSNOR polypeptide (e.g., SEQ ID NO:17-SEQ ID NO:21), peptide (e.g., peptide encoded by SEQ ID NO:9-SEQ ID NO:14), or fragment thereof in a subject, e.g. for diagnostic purposes, are also encompassed by the invention. In accordance with the invention, diagnostic methods can be used to predict or establish the onset of a medical condition described herein, or to monitor the progression or success of treatment of such condition. It is understood that altered expression of polypeptides involved in cell processes and pathways can lead to deleterious effects in a subject. For example, medical conditions that relate to decreased GSNOR levels and increased NO synthesis and/or increased NO levels include, for example, degenerative diseases (e.g., Parkinson's disease, Alzheimer's disease, ALS), stroke (e.g., ischemic stroke), and proliferative diseases (e.g., neoplasms, tumors, cancers, dysplasias, and precancerous lesions). Medical conditions that relate to increased GSNOR levels and decreased SNO levels (e.g., SNO-Hb) include, for example, vascular disorders such as hypertension (e.g., pulmonary hypertension), heart disease, heart failure, heart attack, atherosclerosis, restenosis, asthma, and impotence. Medical conditions that relate to decreased GSNOR levels and increased SNO levels (e.g., SNO-Hb) include, for example, tissue injury (e.g., hepatic, renal, muscle, and/or lymphatic tissue) or death due to systemic infections such as bacteremia, sepsis, systemic inflammatory response syndrome, neonatal sepsis, cardiogenic shock, or toxic shock.

Other conditions that relate to decreased GSNOR levels and increased SNO levels (e.g., SNO-Hb) include, for example, inflammatory disease such as colitis, inflammatory bowel disease, rheumatoid arthritis, osteoarthritis, psoriatic arthritis, infectious arthritis, ankylosing spondylitis, tendonitis, bursitis, vasculitis, fibromyalgia, polymyalgia rheumatica, temporal arteritis, giant cell arteritis, polyarteritis, HIV-associated rheumatic disease syndromes, systemic lupus, erythematosus, gout, and pseudogout (calcium pyrophosphate dihydrate crystal deposition disease), among others. In addition, decreased GSNOR levels and increased SNO levels (e.g., SNO-Hb) are associated with hypotension during anesthesia, and tissue damage and morbidity due to shock (e.g., endotoxic or septic shock), as shown herein below.

One diagnostic method involves providing a biological sample from a subject, measuring the levels of GSNORs or SNOs in the sample, and comparing the level to a reference sample having known GSNOR or SNO levels. A higher or lower level in the sample versus the reference indicates altered expression of GSNORs or SNOs. Alternatively, the enzymatic activity of GSNOR can be measured in any cell of interest. The detection of altered expression or activity of a polypeptide can be use to diagnose a given disease state, and or used to identify a subject with a predisposition for a disease state. Any suitable reference sample may be employed, but preferably the test sample and the reference sample are derived from the same medium, e.g. both are blood or urine, etc. The reference sample should be suitably representative of the level polypeptide expressed in a control population.

The invention also provides a kit to determine GSNOR or SNO levels or GSNOR activity. In one aspect, the kit comprises one or more antibodies directed to a GSNOR polypeptide or peptide, or one or more antibodies directed to a SNO. In another aspect, the kit can contain a substrate for a GSNOR enzyme. Such kits can contain, for example, reaction vessels, reagents for detecting GSNOR or SNO in sample, and reagents for development of detected GSNOR or SNO, e.g. a secondary antibody coupled to a detectable marker. The label incorporated into the anti-polypeptide antibody may include, e.g., a chemiluminescent, enzymatic, fluorescent, colorimetric, or radioactive moiety. For detecting GSNOR enzyme activity, the kit can contain a colormetric or fluorometric assay for measuring reaction with a substrate. As an alternative approach, the kit can include nucleic acid probes for measuring levels of GSNOR gene expression or gene dosage. The nucleic acid probes may be unlabeled or labeled with a detectable marker. If unlabeled, the nucleic acid probes may be provided in the kit with labeling reagents. Kits of the present invention may be employed in diagnostic and/or clinical screening assays.

### Screens for Modulating Agents

The invention further encompasses agents (e.g., inhibitors/antagonists or activators/agonists) which modulate the levels of one or more GSNORs or SNOs, or modulate GSNOR activity, and methods for identifying such agents. Screening assays can be designed to identify compounds that bind or complex with a GSNOR polypeptide or peptide, or otherwise alter expression or stability of the GSNOR transcript or translation product, or interfere with the interaction of GSNOR with other cellular proteins.

The screening assays of the invention can include methods amenable to high-throughput screening of chemical libraries, making them particularly suitable for identifying small molecule drug candidates. The assays can be performed in a variety of formats, including protein-protein binding assays, biochemical screening assays, immunoassays, and cell-based assays, which are well characterized in the art. For in vitro screening, modulating agents can be identified by, e.g., phage display, GST-pull down, FRET (fluorescence resonance energy transfer), or BIAcore (surface plasmon resonance; Biacore AB, Uppsala, Sweden) analysis. For in vivo screening, agents can be identified by, e.g., yeast two-hybrid analysis, co-immunoprecipitation, co-localization by immunofluorescence, or FRET.

Modulation of activity (or levels) due to the test agent, e.g. binding of the agent to the polypeptide, can be determined using art recognized methods. For example, the polypeptide can be detected using polypeptide-specific antibodies, as described above. Bound agents can alternatively be identified by comparing the relative electrophoretic mobility of polypeptides exposed to the test agent to the mobility of complexes that have not been exposed to the test agent. GSNO reductase activity can be measured by GSNO-dependent NADH consumption as previously described (Liu et al., 2001). SNO levels can be measured by photolysis-chemiluminescence (Liu et al., 2000b).

In one specific embodiment, a binding complex between a GSNOR polypeptide and test agent is isolated or detected in the reaction mixture. For example, the GSNOR polypeptide or the test agent can be immobilized on a solid phase, e.g., on a microtiter plate, by covalent or non-covalent attachments. Non-covalent attachment can be accomplished by coating the solid surface with a solution of the GSNOR polypeptide and drying. Alternatively, an immobilized antibody, e.g., a monoclonal antibody, specific for the GSNOR polypeptide to be immobilized can be used to anchor it to a solid surface.

The assay can be performed by adding the non-immobilized component (e.g., the polypeptide or test agent), which may be labeled by a detectable label, to the immobilized component on the solid surface. When the reaction is complete, the non-reacted components can be removed, e.g., by washing, and complexes anchored on the surface can be detected by their label. Where the originally non-immobilized component does not carry a label, complexing can be detected, for example, by using a labeled antibody that specifically binds to the immobilized complex.

If the test agent interacts with a GSNOR polypeptide, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system, e.g., a two-hybrid system (Fields and Song, Nature (London), 340:245-246 (1989); Chien et al., Proc. Natl. Acad. Sci. USA, 88:9578-9582 (1991); Chevray and Nathans, Proc. Natl. Acad. Sci. USA, 89:5789-5793 (1991)). Two-hybrid systems employs two fusion proteins, one in which the target protein is fused to a DNA-binding domain, and another, in which candidate binding proteins are fused to the activation domain (e.g., GAL4 binding and activation domains can be used). Cells are transformed with both fusion constructs, and colonies containing interacting polypeptides are detected with a chromogenic substrate for β-galactosidase. A complete kit (MATCHMAKER™) for identifying protein-protein interactions between two specific proteins using the two-hybrid technique is commercially available from CLONTECH.

Test agents that interfere with the interaction of a GSNOR polypeptide and other intra- or extracellular components can be tested by established methods. In one approach, a reaction mixture is prepared containing the GSNOR gene product and the intra- or extracellular component under conditions and for a time allowing for the interaction and binding of the two products. The reaction is run in the absence and in the presence of the test compound. In addition, an nonreactive agent may be added to a third reaction mixture, to serve as positive control. The formation of a complex in the control reaction(s) but not in the reaction mixture containing the test compound indicates that the test compound interferes with the interaction of the test compound and its reaction partner.

To identify inhibitors, the GSNOR polypeptide may be added to a cell along with the test agent, and then checked for decreased activity. The gene encoding the agent can be identified by numerous methods known to those of skill in the art, for example, ligand panning, FACS sorting, and expression cloning (see, e.g., Coligan et al., Current Protocols in Immun., 1(2): Chapter 5 (1991)). As an alternative approach, labeled GSNOR polypeptide can be photoaffinity-linked with cell membrane or extract preparations that express the receptor molecule. Cross-linked material can be resolved by PAGE and exposed to X-ray film. The labeled complex containing the receptor can be excised, resolved into peptide fragments, and subjected to protein micro-sequencing. The amino acid sequence obtained from micro-sequencing can be used to design a set of degenerate oligonucleotide probes to screen a cDNA library to identify the gene encoding the agent.

One method of identifying an agent (i.e., an inhibitor) which decreases the levels and/or activity of a GSNOR comprises: (a) providing a GSNOR polypeptide or peptide; (b) contacting the GSNOR polypeptide or peptide with a test agent; and (c) detecting the presence of an agent that binds to the GSNOR polypeptide or peptide, wherein the binding agent down-regulates the level and/or activity of the GSNOR polypeptide or peptide. One method of identifying an agent (i.e., an activator) which decreases the levels and/or activity of a GSNOR comprises: (a) providing a GSNOR polypeptide or peptide; (b) contacting the GSNOR polypeptide or peptide with a test agent; and (c) detecting the presence of an agent that binds to the GSNOR polypeptide or peptide, wherein the binding agent up-regulates the level and/or activity of the GSNOR polypeptide or peptide.

In addition, one method of identifying an agent (i.e., inhibitor) which decreases S-nitrosylation comprises: (a) culturing a first cell capable of S-nitrosylation in a media comprising a test agent; (b) culturing a second cell capable of S-nitrosylation in a media without the test agent, wherein the second cell is similar to the first cell except for lacking the test agent; and (c) comparing S-nitrosylation in both the first cell and the second cell wherein the agent which inhibits S-nitrosylation is identified when S-nitrosylation is less in the first cell than in the second cell. One method of identifying an agent (i.e., activator) which increases S-nitrosylation comprises: (a) culturing a first cell capable of S-nitrosylation in a media comprising a test agent; (b) culturing a second cell capable of S-nitrosylation in a media without the test agent, wherein the second cell is similar to the first cell except for lacking the test agent; and (c) comparing S-nitrosylation in both the first cell and the second cell wherein the agent which increases S-nitrosylation is identified when S-nitrosylation is greater in the first cell than in the second cell.

Any compound or other molecule (or mixture or aggregate thereof) can be used as a test agent. In some embodiments, the agent can be a small peptide, or other small molecule produced by combinatorial synthetic methods known in the art. In other embodiments, the agent can be a soluble receptor, receptor agonist, antibody, or antibody fragment. An agent can be a nucleic acid, such as an antisense molecule or interfering RNA molecule which binds to a GSNOR transcript or gene sequence. Agents can be antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments.

For use with the invention, an inhibitor may be a closely related protein, for example, a mutated form of the GSNOR polypeptide that recognizes one or more substrates but lacks enzymatic activity. An inhibitor can be an antisense RNA or DNA construct prepared using antisense technology (described above). Inhibitors can include small molecules that bind to the substrate binding site or other relevant binding site of the GSNOR polypeptide, thereby blocking the normal biological activity. Examples of small molecules include, but are not limited to, small peptides or peptide-like molecules, preferably soluble peptides, and synthetic non-peptidyl organic or inorganic compounds. These small molecules can be identified by any one or more of the screening assays discussed hereinabove and/or by any other screening techniques which are known to those skilled in the art.

### Pharmaceutical Compositions

The invention further encompasses pharmaceutical compositions useful as prophylaxes or treatments (e.g., for alleviating one or more symptoms) for medical conditions. As non-limiting examples, medical conditions that relate to decreased GSNOR levels and increased NO synthesis and/or increased NO levels include degenerative diseases (e.g., Parkinson's disease, Alzheimer's disease, ALS), stroke (e.g., ischemic stroke), and proliferative diseases (e.g., cancers, tumors, dysplasias, and neoplasms). Medical conditions that relate to increased GSNOR levels and decreased SNO levels (e.g., SNO-Hb) include, for example, vascular disorders such as hypertension (e.g., pulmonary hypertension), heart disease, heart failure, heart attack, atherosclerosis, restenosis, asthma, and impotence. Medical conditions that relate to decreased GSNOR levels and increased SNO levels (e.g., SNO-Hb) include, for example, tissue injury (e.g., liver, kidney, muscle, and or lymph tissue) or death due to systemic infections such as bacteremia, sepsis, systemic inflammatory response syndrome, neonatal sepsis, cardiogenic shock, or toxic shock.

Other conditions that relate to decreased GSNOR levels and increased SNO levels (e.g., SNO-Hb) include, for example, inflammatory disease such as colitis, inflammatory bowel disease, rheumatoid arthritis, osteoarthritis, psoriatic arthritis, infectious arthritis, ankylosing spondylitis, tendonitis, bursitis, vasculitis, fibromyalgia, polymyalgia rheumatica, temporal arteritis, giant cell arteritis, polyarteritis, HIV-associated rheumatic disease syndromes, systemic lupus, erythematosus, gout, and pseudogout (calcium pyrophosphate dihydrate crystal deposition disease). In addition, decreased GSNOR levels and increased SNO levels (e.g., SNO-Hb) are associated with hypotension (e.g., in association with anesthesia), and tissue damage and death due to shock (e.g., endotoxic or septic shock), as shown herein below.

In one aspect, the pharmaceutical composition includes a reagent of the invention, which can be administered alone or in combination with the systemic or local co-administration of one or more additional agents. A reagent of the invention can include a GSNOR polypeptide (e.g., SEQ ID NO:17-SEQ ID NO:21), peptide (e.g., a peptide encoded by SEQ ID NO:9-SEQ ID NO:14), an anti-GSNOR antibody or antibody fragment, a GSNOR mimetic (e.g., peptide, small molecule, or anti-idiotype antibody), a GSNOR antisense or iRNA sequence, or fragment, derivative, or modification thereof, or another GSNOR inhibitor or activator. Additional agents for administration may include preservatives, anti-stress medications, phosphodiesterase inhibitors, iNOS inhibitors, β-agonists, and anti-pyrogenics. Suitable phosphodiesterase inhibitors include, but are not limited to, rolipram, cilomilast, roflumilast, Viagra® (sildenifil citrate), Cialis® (tadalafil), Levitra® (vardenifil). Suitable β-agonists include, but are not limited to, isoproterenol, metaproterenol, terbutaline, albuterol, bitolterol, ritodrine, dopamine, and dobutamine.

Suitable iNOS inhibitors include, but are not limited to, Type II iNOS inhibitors, specific NOS inhibitors, and non-specific NOS inhibitors. Non-limiting examples of NOS inhibitors include L-N(6)-(1-iminoethyl)lysine tetrazole-amide (SC-51); aminoguanidine (AG); S-methilisourea (SMT); S-(2-Aminoethyl)isothiourea; 2-Amino-5,6-dihydro-6-methyl-4H-1,3-thiazine (AMT); L-2-Amino-4-(guanidiooxy)butyric acid (L-Canavanine sulphate); S-Ethylisothiourea (EIT); 2-Iminopiperidine; S-Isopropylisothiourea; and 1,4-phenylenebis(1,2-ethanediyl)-diisothiourea (PBIT). Preferred NOS inhibitors for use with the invention are N-[3-(aminomethyl)benzyl]acetamidine (1400W); N6-(1-Iminoethyl)-L-lysine (L-NIL); monomethyl arginine (e.g., for non-specific inhibition); 7-Nitroindazole (e.g., for inhibition of nNOS in brain tissue), etc.

A pharmaceutical composition of the invention is preferably formulated to be compatible with its intended route of administration. Examples of routes of administration include oral and parenteral, e.g., intravenous, intradermal, subcutaneous, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active reagent (e.g., polypeptide, peptide, antibody, or antibody fragment) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams as generally known in the art. The reagents can also be prepared in the form of suppositories (e.g., with conventional suppository bases such as cocoa butter and other glycerides) or retention enemas for rectal delivery.

In one embodiment, the active reagents are prepared with carriers that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the invention are dictated by and directly dependent on the unique characteristics of the active reagent and the particular therapeutic effect to be achieved, and the limitations inherent in the art of compounding such an active agent for the treatment of individuals.

Nucleic acid molecules encoding a proteinaceous agent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral or adenoviral vectors, the pharmaceutical preparation can include one or more cells that produce the gene delivery system.

In one embodiment, the reagent is administered in a composition comprising at least 90% pure reagent. Preferably the reagent is formulated in a medium providing maximum stability and the least formulation-related side effects. In addition to the reagent, the composition of the invention will typically include one or more protein carrier, buffer, isotonic salt and stabilizer. In some instances, the reagent can be administered by a surgical procedure implanting a catheter coupled to a pump device. The pump device can also be implanted or be extracorporally positioned. Administration of the reagent can be in intermittent pulses or as a continuous infusion.

A reagent can be administered in a manner as to pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477). Alternatively, devices can be used for injection to discrete areas of the brain (see, e.g., U.S. Pat. Nos. 6,042,579; 5,832,932; and 4,692,147).

Modifications can be made to the agents to affect solubility or clearance of an amino acid sequence (e.g., polypeptide, peptide, antibody, or antibody fragment). Peptidic molecules may also be synthesized with D-amino acids to increase resistance to enzymatic degradation. In some cases, the composition can be co-administered with one or more solubilizing agents, preservatives, and permeation enhancing agents. The composition can include a preservative or a carrier such as proteins, carbohydrates, and compounds to increase the density of the pharmaceutical composition. The composition can also include isotonic salts and redox-control agents. In addition, the pharmaceutical compositions can be included in a container, pack, or dispenser together with instructions for administration.

In various embodiments of the invention, suitable in vitro or in vivo assays are performed to determine the effect of a specific reagent and whether its administration is indicated for treatment of the affected tissue. Reagents for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Therapeutic Methods

The invention also encompasses methods of preventing or treating (e.g., alleviating one or more symptoms of) medical conditions through use of one or more of the disclosed reagents. A reagent for use with these methods can include a GSNOR polypeptide (e.g., SEQ ID NO:17-SEQ ID NO:21) or peptide (e.g., peptide encoded by SEQ ID NO:9-SEQ ID NO:14), an anti-GSNOR antibody or antibody fragment, a GSNOR mimetic (e.g., peptide, small molecule, or anti-idiotype antibody), a GSNOR antisense or iRNA sequence, or a fragment, derivative, or modification thereof, or another GSNOR inhibitor or activator. As discussed above, altered levels of GSNORs, NO, and SNOs have been implicated in various medical conditions. Thus, methods are disclosed for treating or preventing a disease or disorder involving altered or unwanted levels of GSNORs, NO, and/or SNOs, or GSNOR activity, by administering to a subject a therapeutically effective amount of at least one molecule that modulates the activity or levels thereof.

In subjects with deleteriously high levels of GSNOR or GSNOR activity), modulation may be achieved, for example, by administering a reagent that disrupts or down-regulates GSNOR function, or decreases GSNOR levels (e.g., through decreased production or increased degradation or instability). These reagents may include anti-GSNOR antibodies or antibody fragments, GSNOR antisense, iRNA, or small molecules, or other inhibitors, alone or in combination with other agents (e.g., phosphodiesterase inhibitors) as described in detail herein.

In subjects with deleteriously low levels of GSNOR or GSNOR activity (and concomitantly high levels of SNOs and or NO), modulation may be achieved, for example by administering a reagent that activates or enhances GSNOR function, increases GSNOR levels (e.g., through increased production or stability or decreased degradation), or decreases SNO or NO levels. These reagents may include GSNOR polypeptides or peptides, GSNOR mimetics (e.g., peptides, small molecules, or anti-idiotype antibodies), GSNOR expression vectors, or other activators, alone or in combination with anti-SNO antibodies or antibody fragments, or NOS inhibitors or NO scavengers.

Pharmaceutical preparations suitable for administration of these reagents are described above. Additional agents for administration may include preservatives, anti-stress medications, phosphodiesterase inhibitors, iNOS inhibitors, and anti-pyrogenics as described in detail herein.

In one embodiment, the modulatory method of the invention involves contacting a cell with an agent that modulates one or more of the activities of GSNOR or NOS activity. In another embodiment, the agent stimulates or inhibits the activity of the GSNOR or NOS signaling pathway. These modulatory methods can be performed in vitro (e.g., by culturing the cell with the agent) or, alternatively, in vivo (e.g., by administering the agent to a subject). As such, the invention provides methods of treating an individual afflicted with a disorder, as described above. In one embodiment, the method involves administering a reagent, or combination of reagents that modulate (e.g., up-regulate or down-regulate) GSNOR or NO levels or activity.

As demonstrated herein below, inhibitors of GSNOR may be used as a means to improve β-adrenergic signaling. In particular, inhibitors of GSNOR alone or in combination with β-agonists could be used to treat or protect against heart failure, or other vascular disorders such as hypertension and asthma. GSNOR inhibitors can be used to modulate G protein coupled receptors (GPCRs) by potentiating Gs G-protein, leading to smooth muscle relaxation (e.g., airway and blood vessels), and by attenuating Gq G-protein, and thereby preventing smooth muscle contraction (e.g., in airway and blood vessels).

### SNO-Based Diagnostics and Therapeutics

The invention further encompasses methods of diagnosis and treatment based on measurement or alteration, respectively, of SNO levels in a patient in accordance with the methods disclosed herein (see, e.g., J. Stamler. Circ. Res., 2004; 94: 414-417). One physiological benefit of SNOs as compared to NO is their resistance to inactivation by superoxide (O₂⁻). In damaged tissues, increased O₂⁻ can react with NO to produce toxic peroxynitrite. But the amounts of peroxynitrite that accrue depend at a minimum on the relative rates of NO/O₂⁻ production: NO>O₂⁻ in fact favors production of SNO (Schrammel A, et al., Biol. Med. 2003; 34:1078-1088). Researchers have demonstrated that superoxide, generated by ischemia/reperfusion (I/R) in mesenteric vessels, facilitates the synthesis of SNO-albumin (Ng ESM, et al., Circ. Res. 2004; 94:559-565). SNO-albumin is known to protect tissues against I/R-induced damage (Hallstrom S, Circulation. 2002; 105:3032-3038). Thus, there appears to be a means to exploit superoxide to preserve NO bioactivity. A remaining problem is that the oxidative damage caused by I/R impairs NO production. It is therefore noteworthy that it has also been shown that the thiols of albumin can transport inhaled NO to the gut and subserve relaxation of blood vessels (Ng ESM, et al., Circ. Res. 2004; 94:559-565).

Although relatively high concentrations of SNO-albumin are required to increase blood flow, the amounts that attenuate vasoconstriction are in the physiological range. Furthermore, as evident from the accrual of SNO-albumin in some hypertensive and uremic patients, it is the efficiency of NO group release that determines bioactivity (Tyurin V A, et al., Circ. Res. 2001; 88:1210-1215; Massy Z A, et al., J. Am. Soc. Nephrol. 2004; 15:470-476). In particular, increases in plasma SNO-albumin are associated with high blood pressure and predict adverse cardiovascular outcome (Massy Z A, et al., J. Am. Soc. Nephrol. 2004; 15:470-476). New genetic evidence makes it clear that SNOs play essential roles in the vasculature (Liu L, et al., 2004, Cell 116:617-628). Taken together, these studies suggest that SNO-albumin may dispense NO bioactivity in states characterized by NO deficiency. They also indicate that cysteines in albumin and other key blood proteins such as hemoglobin represent new therapeutic targets.

Inhaled NO increases circulating levels of SNO-albumin, but it does not reveal the mechanism by which SNO-albumin is made, where in the circulation it is produced, or how much NO actually takes this path. Inhaled NO first accumulates in the airways and lung parenchyma in the form of SNOs and other complexes with proteins, and then leaches into the blood (Simon D I, et al., Proc. Natl. Acad. Sci USA 1996; 93:4736-4741; McCarthy T J, et al., Nucl. Med. Biol. 1996; 23:773-777; McCarthy T J, et al., Nucl. Med. Biol. 1996; 23:773-777). Salient features of this process are not currently known, including the form in which NO bioactivity enters the blood over time and the flux through SNO-albumin.

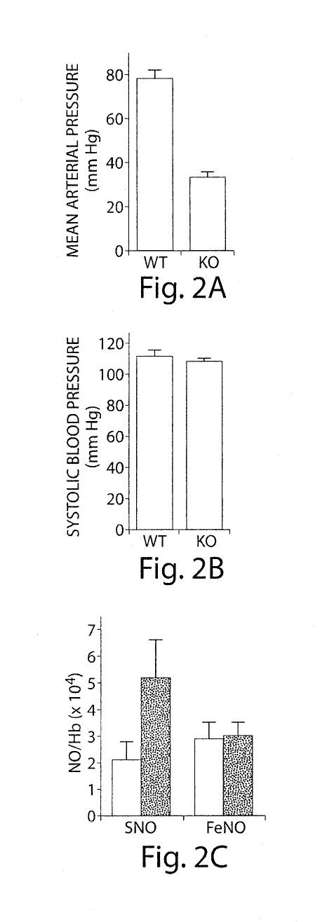

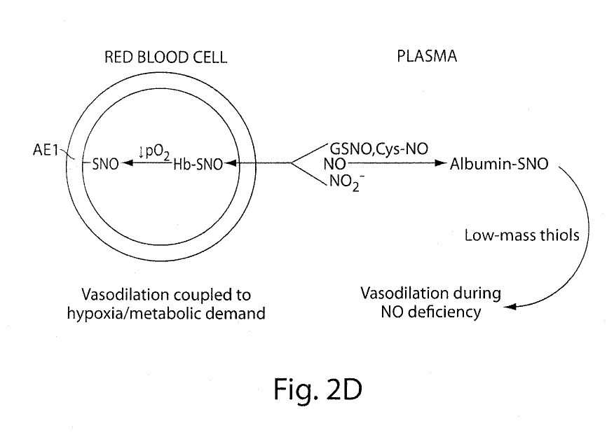

In albumin, both a hydrophobic pocket and bound metals (copper and perhaps heme) can facilitate S-nitrosylation by NO, while hemoglobin has several channels through which it can react with NO, nitrite, or GSNO to produce SNO-Hb (FIG. 2D; see above). It is believed that hemoglobin out-competes albumin for NO. However, this outcome appears not to be absolute, as the relative yield of NO bound to hemoglobin in bioactive form appears inversely proportional to the rate and amount of NO administered and exhibits a plateau at low micromolar levels (Gow A J, Stamler J S, Nature 1998; 391:169-173). By comparison, only nanomolar levels are required for vasoregulation. With the high amounts of NO administered clinically and by other experiments (Ng ESM, et al., Circ. Res. 2004; 94:559-565) it appears that hemoglobin slows the production of SNO by lodging NO on the α-hemes and eliminating it as nitrate (Gow A J, Stamler J S, Nature 1998; 391:169-173; Gow A J, et al., Proc. Natl. Acad. Sci. USA 1999; 96:9027-9032; Luchsinger B P, et al., Proc. Natl. Acad. Sci. USA. 2003; 100:461-466; Napoli C, et al., Proc. Natl. Acad. Sci. USA 2002; 99:1689-1694; Kirima K, et al., Am. J. Physiol. Heart Circ. Physiol. 2003; 285:H589-H596; Gow A J, et al., Proc. Natl. Acad. Sci. USA 1999; 96:9027-9032; Romeo A A, et al., J. Am. Chem. Soc. 2003; 125:14370-14378; Cannon R O 3rd, et al., J. Clin. Invest. 2001; 108:279-287).

In accordance with the present invention, preferred diagnostic assays for SNO levels preserve the physiological milieu, and employ standards that best emulate the molecules being measured (see, Stamler, J. S., 2004, Cir. Res. 94:414-417). Diagnostic assays for determining plasma levels of SNO levels are preferred. Particularly preferred are photolysis/chemiluminescence-based methods as disclosed herein below (see also J. S. Stamler and M. Feelisch, in Methods in Nitric Oxide Research, J. S. Stamler and M. Feelisch, Eds. Wiley, Chichester, UK, 1996, pp. 521-539; Stamler, J. S., et al., 1997, Science 276, 2034-2037; Mannick, J. B., et al., 1999, Science 284, 651-654; Buga, G. M., et al., 1998, Am. J. Physiol. 275, R1256-R1264). Also preferred is the use of stable nitrosothiol standards for, e.g., SNO-albumin or SNO-Hb measurements, in conjunction with such methods. In this way, the range of SNO bond quantum yields can be covered. In addition, dose dependence and reproducibility of assays can be checked to ensure against systematic artifacts. For use with the invention, any biological sample can be used to measure SNO levels, although blood samples are preferred (e.g., serum, plasma, or whole blood), and plasma samples are particularly preferred.

In one aspect, the diagnostic or monitoring method of the invention comprises (a) measuring levels of SNOs in a biological sample from a patient (e.g., plasma levels); (b) comparing the levels of SNOs in the biological sample to levels in a control sample; and (c) determining if the levels of SNOs in the biological sample are higher than the levels of SNOs in the control sample. This method can be used for diagnosing or monitoring medical conditions (or the efficacy of treatments of medical conditions) associated with increased or otherwise deleteriously high levels of SNOs. For example, increased levels of SNO-Hb are associated with hypotension, sepsis, and other conditions as described in detail herein, while increased levels of SNO-albumin are associated with hypertension, preeclampsia, and other conditions with platelet-aggregation.

In another aspect, the diagnostic or monitoring method comprises (a) measuring levels of SNOs in a biological sample from a patient (e.g., plasma levels); (b) comparing the levels of SNOs in the biological sample to levels in a control sample; and (c) determining if the levels of SNOs in the biological sample are lower than the levels of SNOs in the control sample. Such method can be used for diagnosing or monitoring medical conditions (or the efficacy of treatments of medical conditions) associated with decreased or otherwise deleteriously low levels of SNOs. For example, decreased levels of SNO-Hb are associated with heart failure, diabetes, and other conditions (e.g., oxygen deficit conditions) as described herein, while decreased levels of SNO-albumin are associated with renal disease such as uremia and other conditions having defective platelet-aggregation.

In accordance with the invention, the disclosed methods can be used for preventing or treating (e.g., alleviating one or more symptoms of) medical conditions associated with altered or deleterious levels of SNOs through use of one or more of the disclosed reagents. In subjects with increased or deleteriously high levels of SNOs, modulation may be achieved, for example, by administering a reagent (e.g., via intravenous administration) that down-regulates SNO levels. This down-regulation may be achieved by decreasing production or increasing degradation or instability of SNOs, or by increasing activity or levels of GSNOR. Exemplary reagents include GSNOR polypeptides or peptides, GSNOR mimetics (e.g., peptides, small molecules, and anti-idiotype antibodies), GSNOR expression vectors, and other GSNOR activators, as well as anti-SNO antibodies or antibody fragments, small molecules, and other SNO inhibitors, alone or in combination with other agents (e.g., NOS inhibitors or NO scavengers) as described in detail herein. As examples, increased levels of SNO-Hb are associated with hypotension, sepsis, and other conditions as herein described, while increased levels of SNO-albumin are associated with hypertension, preeclampsia, and other conditions with platelet-aggregation. For excess SNOs, treatments can also include infusions of thiols or antioxidants.

In subjects with deleteriously low levels of SNOs, modulation may be achieved, for example by administering a reagent (e.g., via intravenous administration) that up-regulates SNO levels. This up-regulation may be achieved through increasing production or stability or decreasing degradation of SNOs, or by decreasing levels or activity of GSNOR. Exemplary reagents include anti-GSNOR antibodies or antibody fragments, GSNOR antisense, iRNA, small molecules, and other GSNOR inhibitors, as well as SNO activators, alone or in combination with other agents (e.g., phosphodiesterase inhibitors) as described in detail herein. Such methods can be used for medical conditions associated with undesirably low levels of SNOs. As examples, decreased levels of SNO-Hb are associated with heart failure, diabetes, and other conditions (e.g., oxygen deficit conditions) as described herein, while decreased levels of SNO-albumin are associated with renal disease such as uremia and other conditions having defective platelet-aggregation.

### Examples

The examples presented herein below describe the generation of GSNOR-deficient (GSNOR^{-/-}) mice through homologous recombination, and the response of the mice to a nitrosative challenge induced by both LPS and cecal ligation-sepsis. The bacterial endotoxin model of shock was used in the disclosed experiments, since alternative models could obscure the elucidation of the specific roles of SNOs in governance of NO bioactivity. A bacterial model of sepsis was also used. The GSNOR-deficient animals exhibited substantial increases in whole cell S-nitrosylation, tissue damage, and mortality following endotoxic or bacterial challenge. Further, GSNOR−/− mice showed increased basal levels of SNOs in red blood cells and were hypotensive under anesthesia. From the disclosed experiments, it was determined that GSNOR is indispensable for SNO metabolism, for vascular homeostasis, and for survival in endotoxic shock. It was further determined that SNOs regulate innate immune and vascular function, and are actively cleared to ameliorate nitrosative stress. Accordingly, the results obtained herein have identified nitrosylation of cysteine thiols as critical mechanism of NO function in both health and disease.

The examples are presented in order to more fully illustrate the preferred embodiments of the invention. These examples should in no way be construed as limiting the scope of the invention, as defined by the appended claims.

### Example 1

### Experimental Procedures

Construction of a GSNOR Targeting Vector

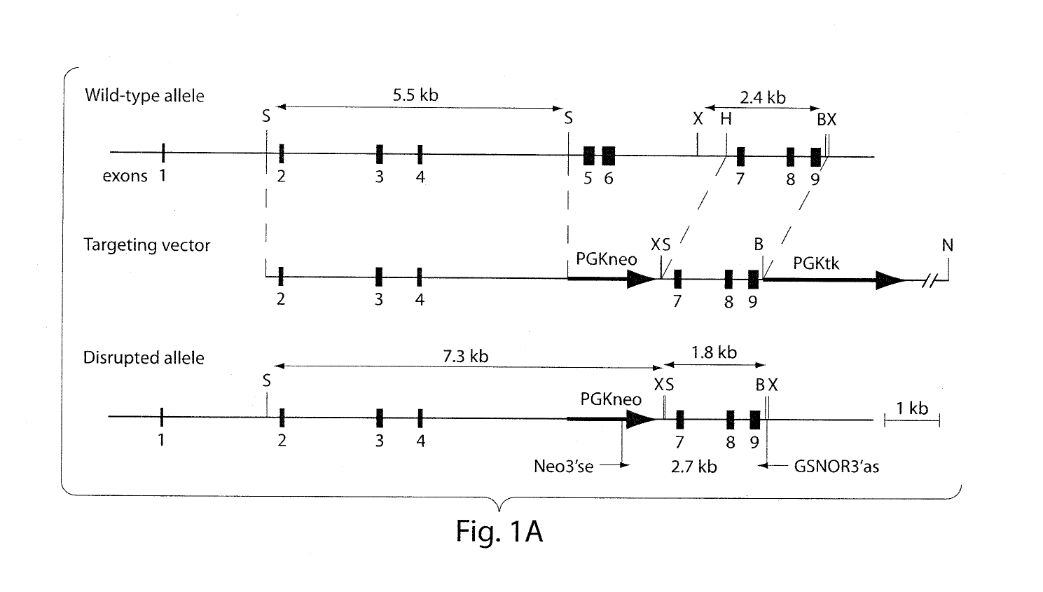

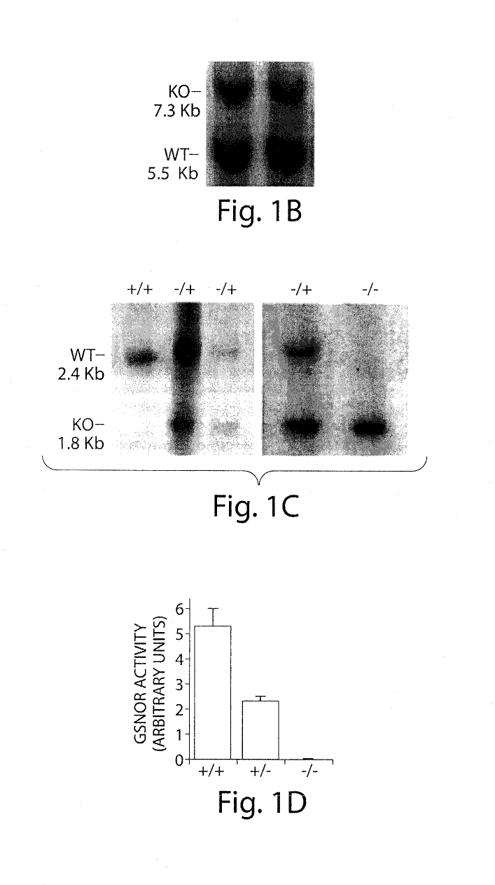

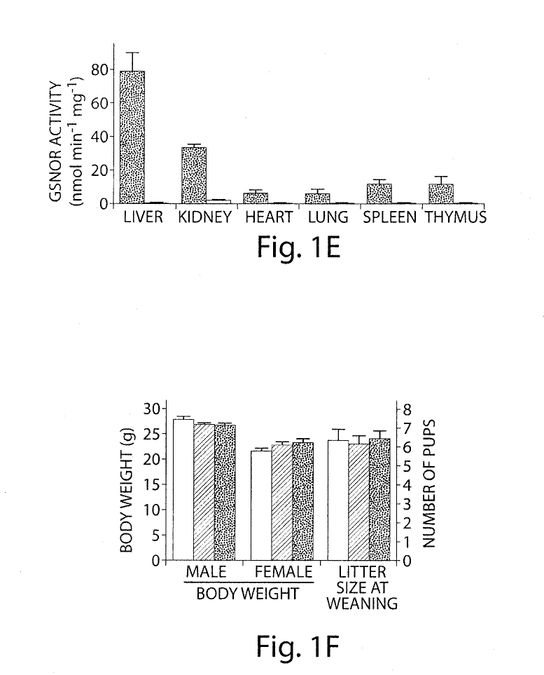

For the disclosed experimental procedures, results, and discussion, see also Liu et al., 2004, Cell 116:617-628, which is incorporated herein by reference in its entirety. For the primers depicted herein, “se” indicates sense strand; “as” indicates antisense strand. A bacterial artificial chromosome (BAC) library derived from genomic DNA of mouse strain 129sv/CJ7 (Invitrogen) was screened for the GSNOR gene by PCR with primers from exon 8 (MoADH1001se, 5′-gatggaagagtgtggagagtg; SEQ ID NO:1) and exon 9 (MoADH1290as, 5′-cagtctcgattatgcacattcc; SEQ ID NO:2) (Foglio and Duester, 1996). Two BAC clones were identified (36c24 and 91m09), and subjected to restriction mapping and Southern blot analysis with probes ex8-9 and ex2-3. The probes were generated from a mouse ADH III cDNA clone (ATCC, GenBank accession number AA008355) by PCR with primer pairs for exons 8-9 (MoADH1001se, MoADH1290as) and exons 2-3 (MoADH52se, 5′-gtgatcaggtgtaaggctgc; SEQ ID NO:3; MoADH295as, 5′-ctgccttcagcttcgtgac; SEQ ID NO:4), respectively. A Sac I fragment containing exons 2-4 and a Hind III-BamH I fragment containing exons 7-9 were isolated from BAC clone 91m09 and inserted 5′ and 3′ to the neomycin resistance gene (neo) in the vector pPNT (Tybulewicz et al., 1991), respectively (FIG. 1A). The resulting GSNOR targeting vector was confirmed by DNA sequencing and linearized by Not I.

Generation of GSNOR^{-/-} Mice

ES cells derived from 129sv mice were transfected with the linearized targeting vector and selected for the presence of neo and absence of the herpes simplex virus thymidine kinase (tk; Duke transgenic mouse facility). Selected ES clones were first screened for homologous recombination by PCR with a neo-derived primer (Neo3′se, 5′-tcttgacgagttcttctgagg; SEQ ID NO:5) and a GSNOR primer (GSNOR3′as, 5′-cagttgactgtcaatgaactgg; SEQ ID NO:6) external to the homologous region in the targeting vector (FIG. 1A). This PCR reaction produced a 2.7 kb DNA fragment only in the cells with the targeted disruption. Recombinant clones were further screened by Southern analyses of Sac I- and Xba I-digested genomic DNA with probes ex2-3 and ex8-9, respectively. The correctly disrupted allele produced a 7.3 kb Sac I and a 1.8 kb Xba I fragment. In contrast, the wild-type allele produced a 5.5 kb Sac I and a 2.4 kb Xba I fragment (FIG. 1A).

Two correctly targeted ES clones with normal karyotype were used independently to generate chimeric mice. These were subsequently bred with C57BL/6 mice to produce F1 heterozygotes. The F1 mice were either mated with each other to produce F2 GSNOR^{-/-} mice or further backcrossed with C57BL/6 mice. Two independent GSNOR^{-/-} mouse lines from the two ES clones were established after both seven and ten consecutive backcrosses with C57BL/6 mice. All mice were fed with standard mouse chow and housed in a pathogen-free facility.

GSNOR Activity

GSNO reductase activity was measured by GSNO-dependent NADH consumption as described previously (Liu et al., 2001).

Blood Pressure

Mice aged 6-8 months were anesthetized by a combination of ketamine (70 mg/kg), xylazine (9 mg/kg), and urethane (1 mg/g). Mean arterial pressure was measured through a catheter inserted in the right carotid artery. Blood pressure was also measured in conscious mice by a computerized tail-cuff system (Krege et al., 1995). Values shown are the means of daily readings on four consecutive days.

Blood Chemistry, Cell Counts, Nitrite, Nitrate and S-nitrosothiols

Blood was obtained by cardiac puncture after animals were euthanized by CO₂ inhalation. The following serum chemistries were quantified by Antech Diagnostics (Farmingdale, N.Y.): alanine aminotransferase (ALT), aspartate aminotransferase (AST), creatine phosphokinase (CPK), urea nitrogen (BUN), creatinine, amylase, lipase, lactate dehydrogenase, alkaline phosphatase, total protein, globulin, albumin, calcium, magnesium, sodium, potassium, chloride, phosphorus, glucose, bilirubin, cholesterol, triglycerides, and osmolality.

The following parameters were measured with a Pentra 60 C+ system of ABX Diagnostics (Montpellier, France): hemoglobin, hematocrit, mean corpuscular volume, and counts of erythrocytes, leukocytes, neutrophils, lymphocytes, monocytes, eosinophils, and platelets.

Levels of iron-nitrosyl hemoglobin and SNO-hemoglobin/SNO-proteins in RBCs were measured by photolysis-chemiluminescence (McMahon et al., 2002).

Serum nitrate and nitrite were measured by capillary electrophoresis (CE) (Zunic et al., 1999) with a P/ACE MDQ system (Beckman) and by chemiluminescence (Sievers NO Analyzer). For CE, sera were diluted (1:10) with water and filtered through a 5 kDa cut-off membrane. Electrophoresis of the filtered samples and of nitrate and nitrite standards was carried out in a neutral capillary with Tris buffer (100 mM, pH 8.0), and monitored by absorbance at 214 nm. Nitrite concentrations are higher when measured by CE than by chemiluminescence (Zunic et al., 1999), but no relative differences between CE and chemiluminescence were observed.

Histology

Organs were fixed with phosphate-buffered formalin and embedded in paraffin. Tissue sections, 5-6 μm thick, were stained with hematoxylin and eosin (H&E). The stained sections were examined by light microscopy by a board certified veterinary pathologist. Apoptosis was assessed by TUNEL assay.

LPS Treatment

LPS (E. coli, serotype 026:B6, Sigma) at a dosage of 150,000 endotoxin units/g (EU/g) was injected intraperitoneally into C57BL/6 and GSNOR^{-/-} mice. The mice were matched for age (11-12 weeks old), gender, and weight. LPS used for the males was lot number 050K4117 (15 million EU/mg) and LPS used for the females was lot number 101K4080 (3 million EU/mg). Studies were done in 45 additional male mice (22 wild-type and 23 GSNOR^{-/-}) administered lot number 101K4080. This ensured that gender and strain differences did not result from a batch effect. Phosphate-buffered saline (PBS, 20 μl/g) was injected in controls. In additional sets of experiments, LPS-challenged GSNOR^{-/-} mice were injected subcutaneously with the iNOS inhibitor 1400W (1 μg/g, Cayman) or PBS (10 μl/g). Injections were performed at 6, 24 and 30 hours after LPS, or at 24, 42 and 48 hours after LPS.

Cecal Ligation and Puncture

Female mice aged 3 months were anesthetized with ketamine (150 mg/kg) and xylazine (10 mg/kg). The cecum was ligated below the ileocecal valve, and punctured once on the anti-mensenteric border with a 26-gauge needle. After surgery, the mice were subcutaneously injected with 0.5 ml of normal saline.

Septic Shock in Humans

Twelve consecutive adult patients with septic shock in the Duke University Medical Center (DUMC) ICU were enrolled. Septic shock was defined according to the American College of Cardiology/Society of Critical Care Medicine guidelines (1992). The presence of gram-negative bacteremia within 72 hours of enrollment was ascertained from the medical records. The control group consisted of 12 healthy volunteers. Radial arterial and central venous blood samples were collected for analysis of RBC NO content (McMahon et al., 2002). Informed consent was obtained, and the study was approved by the DUMC Internal Review Board.

Liver SNO

Liver homogenates were prepared in lysis buffer (20 mM Tris-HCl, pH 8.0, 0.5 mM EDTA, 100 μM diethylenetriamine pentaacetic acid, 0.1% NP-40 and 1 mM phenylmethylsulfonyl fluoride). SNO levels in the total lysate and in a fraction filtered through a 5 kDa cut-off ultrafiltration membrane (low-mass SNO) were measured by photolysis-chemiluminescence (Liu et al., 2000b) and normalized for protein content.

Statistical Analysis

Survival data on day 6 after LPS treatment were analyzed by both the χ² test and the Fisher exact test of contingency tables, with similar results. Blood pressure, SNO levels and serum chemistries were analyzed with the Student's t-test or with the nonparametric Mann-Whitney test.

### Example 2

### Results

Generation of GSNOR^{-/-} Mice

The GSNOR gene includes nine exons (Foglio and Duester, 1996); exons 5 and 6 encode most of the coenzyme-binding domain of GSNOR (Yang et al., 1997). A targeting vector was constructed with GSNOR genomic DNA. This was used to replace exons 5 and 6 with a neomycin resistance gene (neo) through homologous recombination in mouse (129sv) embryonic stem (ES) cells (FIG. 1A). Homologous recombination on both sides flanking the targeted region was confirmed in four ES clones. Southern blot analyses were performed with probes specific to exons 2-3 and exons 8-9, respectively. As further confirmation, PCR was performed to specifically identify the disrupted allele (FIG. 1B).

Two mouse lines with the targeted disruption were independently generated from two of the ES clones (FIG. 1C). Southern hybridization with a probe specific to exons 8-9 showed that GSNOR^{-/-} mice included only a single mutant (1.8 kb) fragment that resulted from recombination. These mice were backcrossed consecutively to C57BL/6 mice a total of seven-ten times. GSNO reductase activity was absent in both tail and tissues of GSNOR^{-/-} mice (FIGS. 1D and 1E). The activity in heterozygous (GSNOR^{+/-}) mice was roughly half that in wild-type litter-mates.

Phenotype

Heterozygous males and females were bred under pathogen-free conditions. This produced 31 (25%) wild-type, 61 (50%) heterozygous and 30 knockout (25%) mice at weaning. Thus, the inheritance of the wild-type and disrupted GSNOR gene followed the expected Mendelian ratio.

TABLE 1
Growth and Reproduction of GSNOR^{-/-} and wild-type mice

Body Weight (g)^{a}

| Genotype | Male | Female | Litter Size^{b} |
|---|---|---|---|
| C57BL/6 | 27.9 ± 0.5 | 21.7 ± 0.4 | 6.4 ± 0.6 |
| GSNOR^{-/-} 1 | 26.8 ± 0.5 | 22.7 ± 0.6 | 6.2 ± 0.4 |
| GSNOR^{-/-} 2 | 26.8 ± 0.5 | 23.4 ± 0.5 | 6.5 ± 0.4 |

GSNOR^{-/-} and C57BL/6 mice were raised on a standard mouse diet in the same animal facility.
^{a}Values are mean ± SD of 80-day-old mice (n = 18-29).
^{b}Values are mean ± SD at weaning (n = 16-32).

The GSNOR-deficient mice did not show a survival disadvantage under these conditions. GSNOR^{-/-} mice reproduced litters with a size and frequency similar to C57BL/6 mice (FIG. 1F). They developed normally and weighed the same as C57BL/6 mice (FIG. 1F). Histological examination of 4 wild-type (2 males, 2 females) and 4 GSNOR^{-/-} mice (2 males, 2 females) showed no gross morphological or histological difference between the two mouse strains in any of the tissues studied. This included brain, heart, lung, liver, kidney, spleen, thymus, mesenteric lymph node, salivary gland, gastrointestinal tract, pancreas, testis, ovary, uterus, and urinary bladder. Blood cell counts and serum chemistries were normal in GSNOR^{-/-} mice (see below).

Blood Pressure and Basal SNO

emodynamic responses to SNOs, although the mechanism has not been unexplained (Travis et al., 1997). As shown herein, blood pressure was much lower in GSNOR^{-/-} mice than in the wild-type mice (P<0.001) when anesthetized with urethane (FIG. 2A). In contrast, blood pressure in conscious GSNOR^{-/-} mice did not differ from the controls (FIG. 2B).

Previous studies have determined that most of the NO in blood is found in red blood cells (RBCs) as iron nitrosyl hemoglobin and SNO-hemoglobin (Jia et al., 1996; Kirima et al., 2003; McMahon et al., 2002; Milsom et al., 2002). Here it is shown that levels of SNO-Hb (RBC-SNO) were higher in unanesthetized GSNOR^{-/-} mice than in wild-type mice (P<0.05). Yet, levels of iron nitrosyl hemoglobin did not differ between wild-type and GSNOR^{-/-} (FIG. 2C). Thus, the experiments disclosed herein indicate that GSNOR deficiency caused increases in basal SNO, and predisposed mice to disregulation of blood pressure. Endotoxin treatment precluded accurate measurement of blood pressure due to diminished and irregular tail blood flow in un-anesthetized mice and low blood pressure in anesthetized mice.

Mortality from Endotoxic Shock

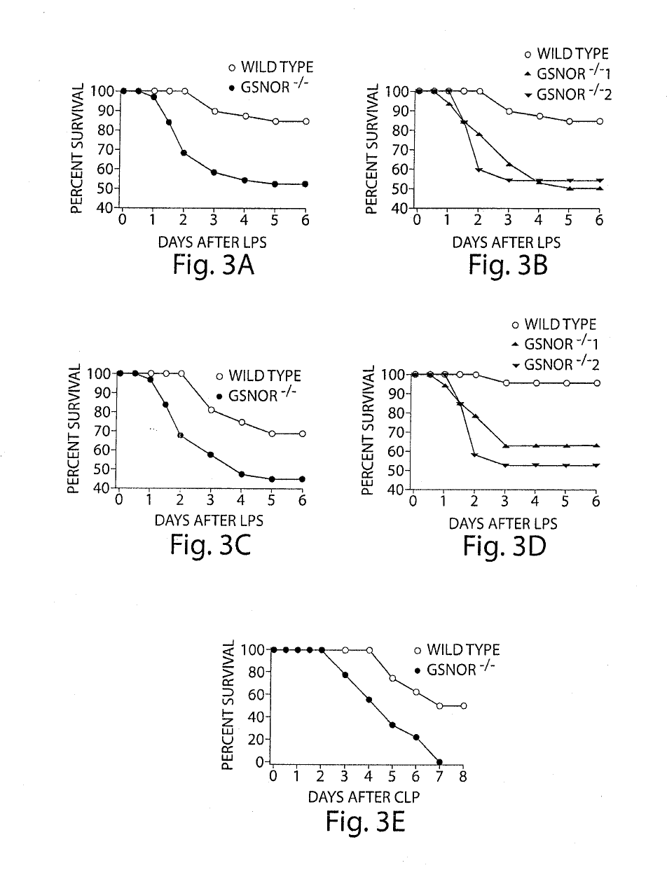

As demonstrated herein, LPS-induced shock was used as a model of nitrosative stress. The dose of LPS for producing ˜50% mortality in GSNOR^{-/-} mice was established in initial dose-response studies (FIGS. 3A-3D). In a larger analysis, this dose of LPS resulted in the death of 48% of GSNOR^{-/-} mice, but only 15% of wild-type mice (FIG. 3A). The difference in mortality between the two strains was determined to be highly significant (P<0.001). Further, both lines of GSNOR-knockout mice, GSNOR^{-/-}1 and GSNOR^{-/-}2, responded similarly to LPS (FIG. 3B), and both succumbed more readily than wild-type mice. Thus, it was highly unlikely that the hypersensitivity of GSNOR^{-/-} mice to LPS resulted from a random mutation created while generating the mice.

In previous studies, the protection conferred by iNOS in endotoxic shock was observed predominantly in female mice (Laubach et al., 1998). The consequence of GSNOR deficiency was therefore studied separately in males (FIG. 3C) and females (FIG. 3D). As shown herein, the LPS dose employed resulted in the death of 37% and 47% of the female GSNOR^{-/-}1 and GSNOR^{-/-}2 respectively, whereas it killed only 4% of wild-type controls (FIG. 3D). The mortality of male GSNOR^{-/-} mice (treated with LPS) was also lower than that of wild-type controls (FIG. 3C), but the difference did not reach statistical significance (P=0.12). The mortality of female wild-type mice (4%) was significantly lower than their male counterparts (29%; P=0.022), but this gender effect was abrogated by GSNOR deletion. It was observed that mortality in female GSNOR^{-/-} mice (42%) was not significantly lower than that of male knockouts (55%; P=0.29). Taken together, these results show that GSNOR clearly protects female mice from endotoxic shock, and suggest that the basis of gender-related resistance to LPS involves GSNOR. Accordingly, female mice were used for most of the studies detailed below.

SNO Metabolism

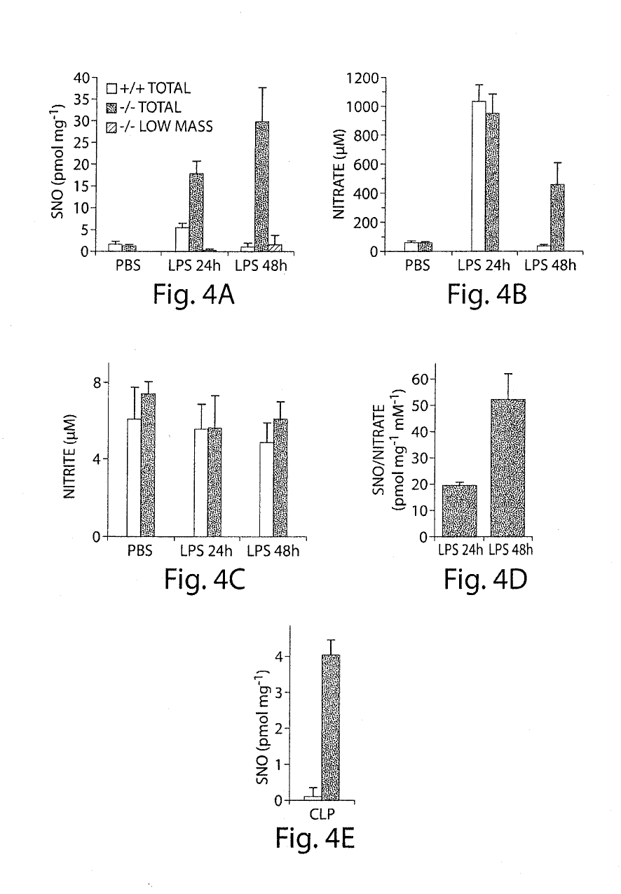

Metabolism of S-nitrosothiols was examined in mouse liver, since this tissue exhibits the highest GSNOR activity in the body (FIG. 1E) (Uotila and Koivusalo, 1997), and expresses substantial iNOS activity during septic shock (Knowles et al., 1990). Hepatic iNOS has been determined to be protective in such situations (Ou et al., 1997). As shown herein, the baseline levels of SNO were similar in GSNOR^{-/-} mice and wild-type mice (FIG. 4A). After i.p. injection of LPS, SNOs in wild-type mice increased modestly at 24 hours (h) and returned to basal levels by 48 h (FIG. 4A). In contrast, SNOs in GSNOR^{-/-} mice increased to high levels at 24 h and increased further at 48 h (FIG. 4A). At 24 h and 48 h time points, SNO levels in the GSNOR^{-/-} mice were, respectively, 3.3-fold and 29-fold greater than in wild-type controls. Over 90% of the SNO could be ascribed to molecules of high mass (>5,000 daltons; FIG. 4A). Thus, in endotoxic shock, the metabolism of endogenously generated nitrosothiols was severely impaired in the GSNOR-deficient mouse.

The levels of nitrate plus nitrite (NOₓ) in the circulation have been known to reflect overall NOS activity in mammals. Here, it was found that basal nitrate levels in GSNOR^{-/-} mice did not differ from wild-type mice (FIGS. 4B, 4C, and Methods). After treatment with LPS, nitrate concentrations in wild-type mice rose at 24 h to the same level as in GSNOR^{-/-} mice, and returned to baseline at 48 h (FIG. 4B). While the nitrate level in GSNOR^{-/-} mice decreased at 48 h (˜50%, P=0.03), it was still substantially elevated above baseline (FIG. 4B). The levels of serum nitrite (<<nitrate) were not significantly different at any time-point in wild-type and mutant animals (FIG. 4C). These data suggested that wild-type and GSNOR^{-/-} mice express equal iNOS activity at 24 h after LPS. By 48 h, iNOS activity returns to baseline in wild-type mice, but the decline in activity is slower in GSNOR^{-/-} mice. This conclusion was confirmed by study of iNOS expression using Western blot analyses of liver lysates.

Although steady-state S-nitrosylation was elevated only in animals with elevated nitrate concentrations, it became clear that SNO levels in tissues were independent of NO (FIGS. 4A-4E). For example, GSNOR^{-/-} mice accumulated much higher amounts of SNO than wild-type mice despite equal levels of nitrate at 24 h after LPS (FIGS. 4A-4B). Furthermore, the ratio of liver SNO to serum nitrate in GSNOR^{-/-} mice was considerably higher at 48 h than at 24 h after LPS (FIG. 4D). Thus, the level of S-nitrosylation in vivo was regulated independently by GSNOR and NOS. Alternatively stated, SNO levels were regulated by both synthesis and turnover and did not correlate directly with amounts of nitrate or nitrite.

Tissue Injury and Recovery Following Endotoxin Challenge

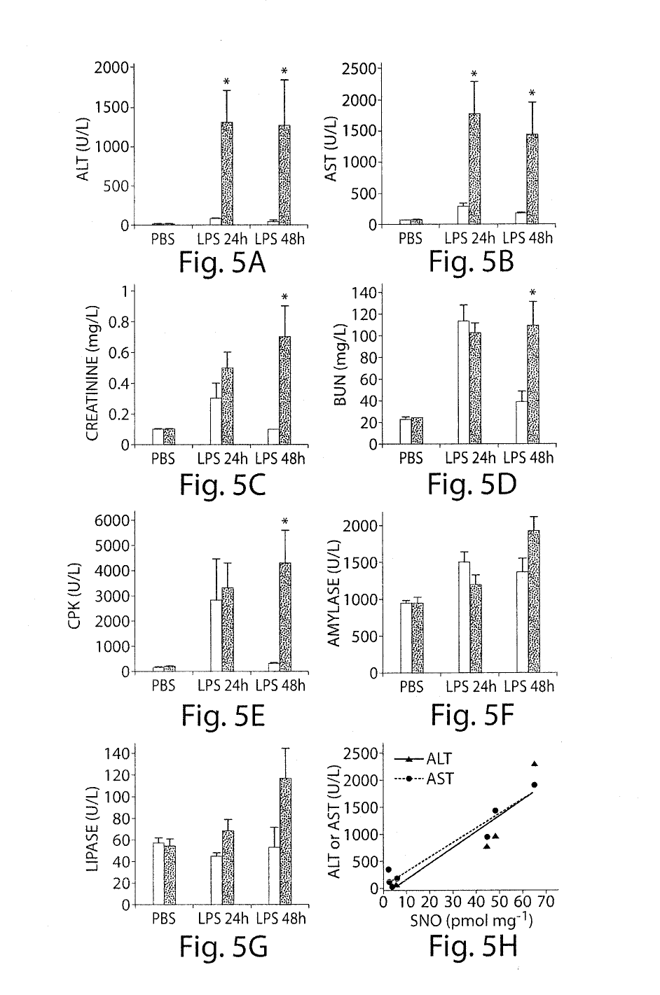

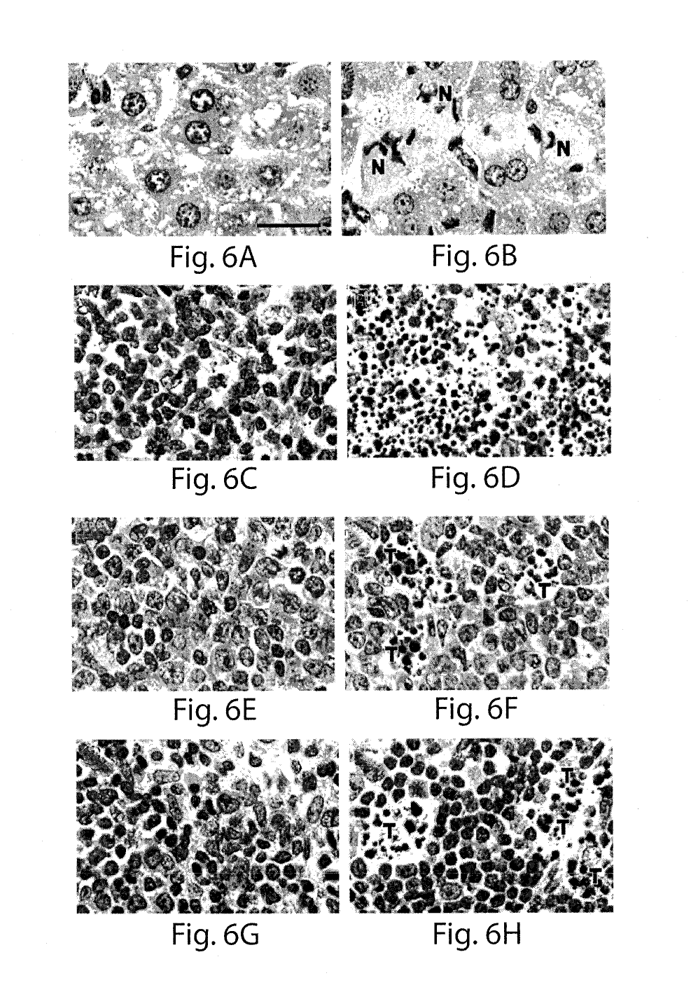

Tissue injury during endotoxic shock was assessed by measurement of serum levels of marker enzymes (FIGS. 5A-5H) and histopathology (FIGS. 6A-6H). In wild-type mice, levels of alanine aminotransferase (ALT) and aspartate aminotransferase (AST), markers of liver injury, increased modestly at 24 h after treatment with LPS and declined by 48 h (FIGS. 5A-5B). By contrast, both ALT and AST increased markedly in the GSNOR^{-/-} mice at 24 h and remained unchanged at 48 h (FIGS. 5A-5B). Increases in ALT and AST were directly correlated with elevations in liver SNO in GSNOR^{-/-} mice (FIG. 5H).

Histological examination of the liver at 24 h (after LPS) showed minimal to mild hepatocellular swelling and cytoplasmic vacuolation in the wild-type mice. By 48 h, the damage had partly resolved (more so in females than males) and no ongoing injury was detected (FIG. 6A). Hepatocellular injury was more severe in the GSNOR^{-/-} mice at 24 h and no recovery was evident at 48 h (FIGS. 6A-6B). At 24 h and 48 h, multifocal necrotic and apoptotic hepatocytes were detected in GSNOR^{-/-} mice (hyaline eosinophilic cytoplasm and pyknotic and karyorrhectic nuclei; FIG. 6B). In addition, GSNOR^{-/-} livers contained disrupted hepatic cords, compressed sinusoids, small aggregates of degenerating granulocytes, and subintimal accumulations of granulocytes and lymphocytes in venules. Thus, both the serum markers and histopathology indicated that LPS-induced liver damage was much worse in GSNOR^{-/-} mice than in wild-type mice. In sharp contrast to the near complete recovery by wild-type livers, GSNOR^{-/-} livers showed no sign of recovery.

The marker of muscle injury, creatine phosphokinase (CPK), and the markers of kidney dysfunction, urea nitrogen (BUN), and creatinine, all increased substantially and to similar levels in wild-type and GSNOR^{-/-} mice at 24 h after LPS (FIGS. 5C-5E). In wild-type controls, these activities decreased at 48 h almost to baseline, but levels did not decline in the GSNOR^{-/-} mice (FIGS. 5C-5E). Thus, organ dysfunction did not resolve in the GSNOR^{-/-} mice. The kidneys and hearts of the wild-type and GSNOR^{-/-} mice were grossly normal on histological examination.

Pancreatic islet cells are known to be highly susceptible to NO toxicity in vitro (Liu et al., 2000a). However, as shown herein, LPS challenge had little effect on the pancreas in wild-type and GSNOR^{-/-} mice. In particular, serum levels of both amylase and lipase changed little following LPS treatment (FIGS. 5F-5G), and no histological abnormalities were detected.

A protective role for GSNOR was evident in lymphatic tissue (FIGS. 6C-6H). At 24 h after LPS, the two strains showed a similar amount and pattern of lymphocyte apoptosis in thymus, spleen, mesenteric lymph nodes, Peyer's patches, and other lymphoid tissues. Wild-type lymphatic tissues showed little cell death at 48 h after LPS (FIGS. 6C, 6E and 6G). In contrast, GSNOR^{-/-} tissues showed substantial apoptosis (FIGS. 6D, 6F and 6H). Lymphocyte apoptosis in the thymus was extensive, especially in cortical regions (FIG. 6D). Further, at 48 h after LPS, lymphocyte depletion was more severe in the GSNOR^{-/-} thymus than in the wild-type (FIGS. 6C-6D). Thus, GSNOR was required to protect the immune system from endotoxic injury.

Effect of iNOS Inhibition on Tissue Injury and Survival of GSNOR^{-/-} Mice

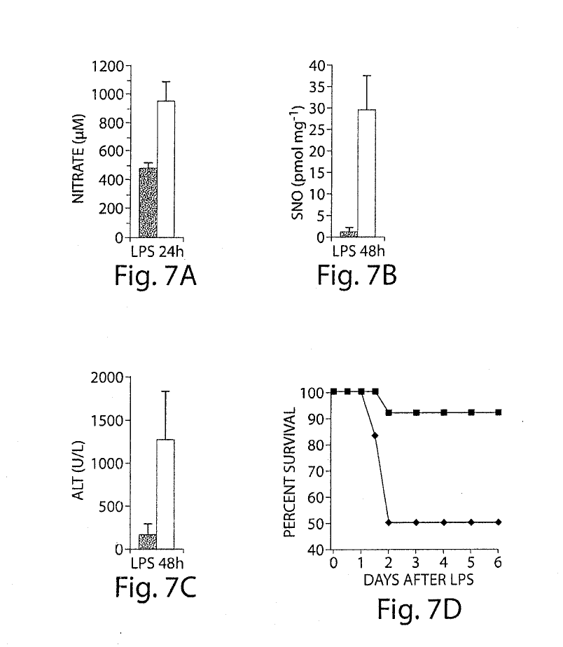

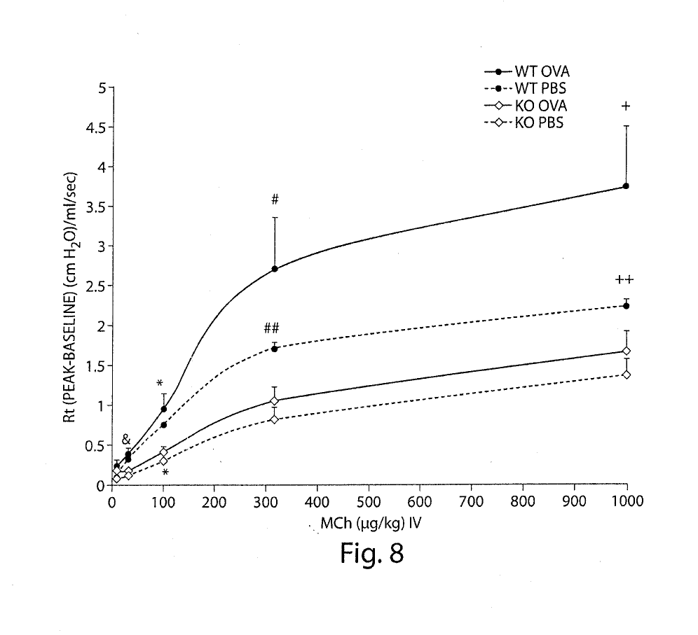

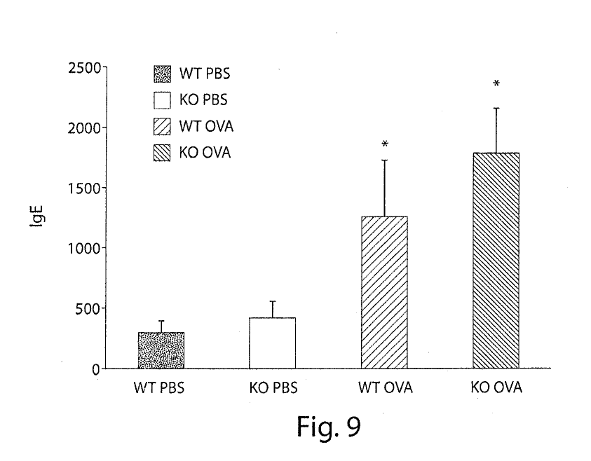

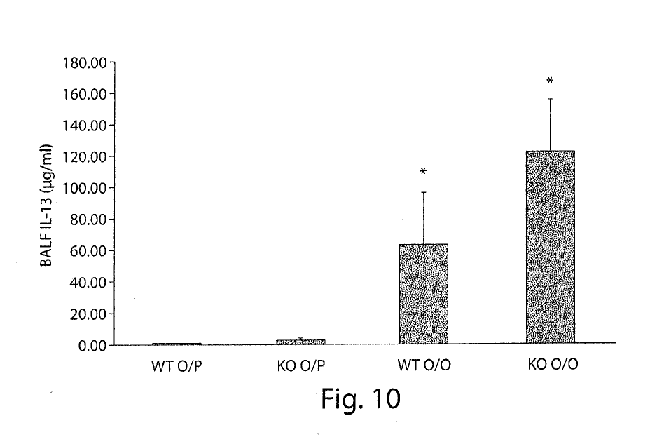

Additional experiments were performed to establish the contribution of nitrosative stress to the pathogenesis of endotoxic shock. LPS-challenged GSNOR^{-/-} mice were treated with 1400 W, a selective iNOS inhibitor. Administration of 1400W, initiated 6 h following LPS injection, reduced serum nitrate (i.e., NOS activity) by about 50% (FIG. 7A, P=0.015) and liver injury by about 90% (FIG. 7C, P=0.020). This improvement coincided with a reduction of liver SNO by about 90% (FIG. 7B). Measurements of serum markers showed that tissue injury was also reduced in kidney, pancreas, and muscle (48 h after LPS treatment). Most importantly, the survival rate of LPS-challenged GSNOR^{-/-} mice was significantly improved by 1400W (FIG. 7D vs. FIG. 3D, P=0.03). By comparison, PBS (volume control) had little effect (FIG. 7D vs. FIG. 3D). When administration of 1400W was delayed for 24 h following LPS injection, this allowed SNOs to accumulate to hazardous levels, and the protection conferred by NOS inhibition was lost (3 out of 5 female GSNOR^{-/-} mice died). These data strongly suggested that nitrosative stress from iNOS in GSNOR^{-/-} mice mediated tissue damage and increased mortality.

GSNOR in Septic Shock

The role and function of GSNOR was also investigated in bacterial septic shock induced by cecal ligation and puncture (CLP) (Wichterman et al., 1980), an animal model that resembles the human condition. CLP resulted in significantly higher mortality in GSNOR^{-/-} mice (n=9) than in wild-type mice (n=8) (FIG. 3E), whereas a sham control without puncture did not result in death of either GSNOR^{-/-} (n=3) or wild-type (n=3) mice. Following CLP, the levels of liver SNOs (FIG. 4E) and marker enzymes were significantly higher in GSNOR^{-/-} than in wild-type mice. Thus, GSNOR protects mice against SNO-related morbidity and mortality induced by CLP.

### Example 3

### Discussion

The disclosed experiments demonstrate that: (1) S-nitrosothiols play an essential role in NO biology, influencing blood pressure and related homeostatic functions, and contributing to the pathogenesis of endotoxic/septic shock; (2) NO bioactivity is regulated not only at the level of synthesis (i.e., NOS) but also by degradation, in particular by GSNOR; (3) turnover of GSNO influences the level of whole cell S-nitrosylation; (4) accumulation of SNOs can produce a stress on the mammalian organism that influences survival, and in particular, nitrosative stress that is identified with GSNO is implicated in disease pathogenesis; (5) GSNOR protects mice from excessive declines in blood pressure under anesthesia, and from tissue injury following endotoxemia; (6) the systems affected most by GSNOR deficiency include the liver, immune system and cardiovascular system. These results signal a fundamental change for the current paradigm of NO biology, which centers on the activity of NOS. The disclosed data also provide genetic support for the importance of redox-based regulation of proteins through modification at cysteine thiols.

GSNOR is Essential for SNO Metabolism

According to the disclosed data, it appears that GSNO reductase is not essential for development, growth, and reproduction of mice. It has been suggested that normal growth and reproduction of ADH III-deficient mice requires dietary supplementation with large amounts of vitamin A (retinol) (Molotkov et al., 2002). Here, no such requirement was observed in either of the GSNOR^{-/-} mouse strains. The origin of the unusual nutritional requirement reported by Molotkov et al. may lie in the deletion construct that was used or in the genetic background of the mice.

One important discovery disclosed herein is that GSNOR is crucial for SNO metabolism in animals. GSNOR^{-/-} mice accumulated higher amounts of S-nitrosothiols than wild-type mice despite comparable levels of NOS expression and activity. Levels of SNOs in vivo were thus determined not only by NOS activities, but also by GSNOR. This conclusion was further supported by the observed increases in GSNOR^{-/-} mice regarding (1) the ratio of SNO to iron nitrosyl compounds at basal conditions; and (2) the ratio of SNO to nitrate or nitrite or both during the course of endotoxic shock. Inasmuch as measurements of nitrite and nitrate are the standard means of assessing NO bioactivity in biological systems, the disclosed results raise interesting questions regarding many previous assumptions.

GSNO is the only SNO substrate recognized by GSNOR, yet the disclosed data indicate deletion of the enzyme results in greater increases in SNO-proteins than in GSNO itself. Similar results were obtained in GSNOR-deficient yeast (Liu et al., 2001) and in RBCs exposed to GSNO ex vivo (Jia et al., 1996). This suggests that at least some key protein SNOs are in equilibrium with GSNO both under basal and stress conditions (Equation 1, below). In addition, the equilibrium apparently favors protein SNOs. Prompt disposal of GSNO by GSNOR (Equation 2, below), acts to drive the equilibrium towards the denitrosylated state. Thus, it appears that glutathione (GSH) cannot effectively or fully terminate SNO signaling or protect proteins from hazardous levels of S-nitrosylation in the absence of GSNOR.

$$\begin{matrix}Protein \text{-} SNO + GSH ⇆ GSNO + protein & Equation 1 \\ GSNO + NADH + {H}^{+} {}_{GSH}^{GSNOR} GSSG + {NH}_{4}^{+ } & Equation 2\end{matrix}$$

GSNOR Protects from Nitrosative Stress in Response to Endotoxin and Bacteria

In the experiments disclosed herein, mice with elevated iNOS activity were subjected to nitrosative stress characterized by elevated levels of S-nitrosylated proteins. However, the LPS-challenged mice did not suffer detrimental consequences unless protection afforded by GSNOR was abolished (GSNOR^{-/-}). In the absence of GSNOR, the animals exhibited hazardous accumulations of S-nitrosylated proteins and tissue damage. The finding herein that GSNOR protected lymphatic tissues and liver from apoptosis added support to the accumulating evidence that death signaling is regulated by SNOs (Eu et al., 2000; Haendeler et al., 2002; Mannick et al., 1999; Marshall and Stamler, 2002; Matsumoto et al., 2003). Additionally, as shown herein, inhibition of iNOS improved all measures of injury across tissues as well as survival of the animals. Collectively these data establish that nitrosative stress is a major cause of morbidity in GSNOR^{-/-} mice.

GSNOR is one of several factors that mediate resistance to microbial challenge (Cohen, 2002). As such, its role is influenced not only by microbial susceptibility to SNOs, but also by its part in protecting immune function (FIGS. 6A-6H). This complexity notwithstanding, recent genetic and chemical evidence suggests that SNOs are produced in mice to counter cryptococcal (de Jesus-Berrios et al., 2003), salmonella (De Groote et al., 1996) and tuberculous (MacMicking et al., 1997) infections. The findings disclosed herein indicate that SNOs are also produced by the host in additional forms of polymicrobial/gram negative sepsis. Specifically, the protection afforded by GSNOR was not only observed in the endotoxic model of shock, but also against CLP-induced bacteremia.

As demonstrated herein, GSNOR deficiency resulted in elevated hepatic levels of SNOs in the CLP model. In support of relevance of these mouse models to the human condition, we find that SNO-Hb levels, which are known to be increased in the blood of endotoxic animals (Jourd'heuil et al., 2000), were several-fold higher in the blood of patients with gram-negative sepsis (0.0037±0.0010 SNO/Hb, n=7) than in healthy controls (0.0010±0.0004 SNO/Hb, n=12; P<0.01). Taken together, these data suggest that S-nitrosothiols may play important roles in both the amelioration and pathogenesis of endotoxic/septic shock.

Gender

GSNOR deficiency resulted in a 10-fold increase in mortality (vs. wild-type) in LPS-challenged female mice, but only a ˜2-fold increase in males. The protective effect of GSNOR may therefore contribute to the relative resistance of females to septic shock. This phenomenon is seen in both animals (Laubach et al., 1998; Zellweger et al., 1997) and humans (Oberholzer et al., 2000; Schroder et al., 1998). Previous experiments showed that iNOS protects female mice more than male mice from endotoxemia-induced death (Laubach et al., 1998). The sum of these data suggests that the beneficial effect of iNOS is nullified by nitrosative stress in GSNOR^{-/-} animals. Without wishing to be bound by theory, it is hypothesized that GSNOR is a genetic determinant of sepsis outcome, particularly in female patients, and that the potential benefits of iNOS inhibition in septic patients will relate to GSNOR activity.

Hemodynamic Consequence of GSNOR Deficiency

Hypotension has been observed as one of the most frequent side effects of anesthesia, but the basis for patient susceptibility has not been determined. Here, GSNOR-deficient mice were hypotensive when anesthetized in the absence of LPS challenge. In GSNOR-deficient animals, basal SNO levels were increased approximately two-fold in RBCs. These levels have been known to produce vasodilation in bioassays (McMahon et al., 2002; Pawloski et al., 2001) and lower blood pressure (or vascular resistance) when either RBCs or SNO-Hb (the major RBC SNO) were infused intravenously (Jia et al., 1996). It was previously shown that urethane or pentobarbital anesthesia markedly potentiates the vasorelaxant and hypotensive effects of SNOs administered intravenously in rats (Travis et al., 1997). The disclosed results point to the possibility that blood pressure under anesthesia may reflect SNO bioactivity and may have a genetic basis in GSNOR activity.

Interestingly, the hypotensive effects of iNOS have also been linked to anesthesia. Hypotension has been found to be greater in pentobarbital-anesthetized wild-type mice challenged with LPS than in iNOS^{-/-} mice (MacMicking et al., 1995). In addition, concentrations of LPS that lowered blood pressure to comparable degrees in anesthetized vs. conscious iNOS^{-/-} mice, produced far greater hypotension in anesthetized than conscious wild-type mice (MacMicking et al., 1995; Rees et al., 1998). LPS has been known to increase levels of SNO-Hb in rodents (Jourd'heuil et al., 2000). Here, similar increases were observed in the blood of patients with sepsis. Collectively, these data suggest that the increased SNOs derived from iNOS contribute to the hypotensive effects of anesthesia in endotoxic animals. The disclosed data do not exclude the effects of GSNO exerted centrally or in the kidney (Ortiz and Garvin, 2003; Stamler, 1999; Stoll et al., 2001). However, the results support the recent discovery that RBCs dilate blood vessels (Gonzalez-Alonso et al., 2002; Jia et al., 1996; McMahon et al., 2002) and the proposition that SNOs in RBCs may contribute to a hypotensive phenotype.

The mechanism(s) by which SNOs are both generated in RBCs and the activity liberated to dilate blood vessels (McMahon et al., 2002; Pawloski et al., 2001) is only partly understood. It has been shown that Hb can react with NO (Gow et al., 1999), nitrite (Luchsinger et al., 2003) or GSNO (Jia et al., 1996; Romeo et al., 2003) to produce SNO-Hb, and that vasodilation by RBCs requires transfer of the NO from SNO-Hb to RBC membrane thiols (Pawloski et al., 2001). Additional studies point to a role for plasma GSNO in dispensing of RBC membrane bioactivity (Lipton et al., 2001). The disclosed results clearly establish the importance of GSNO/GSNOR in maintaining the levels of RBC-SNO in vivo. Moreover, the disclosed experiments show that increases in SNO occur without detectable increases in other bioactive NO compounds (iron nitrosylHb and nitrite). This provides strong genetic support for the idea that SNOs can mediate NO bioactivity in blood (Jia et al., 1996; Stamler et al., 1992) and tissues (Gow et al., 2002; Stamler et al., 2001).

Conclusion

The disclosed findings underscore the central role of S-nitrosothiols in NO biology and disease. Specifically, the genetic evidence provided in this study suggests that GSNO turnover is required not only to prevent accumulation of SNO that predisposes to disease diathesis, but also to regulate the turnover of SNOs in the context of physiological signaling (e.g. the dispensing of a messenger to regulate blood pressure). This homeostatic role of GSNO reductase is reminiscent of that played by superoxide dismutase (SOD). GSNOR affords protection against nitrosative stress and influences vascular tone in a way that is evocative of SOD protection against oxidative stress and regulation of blood pressure (Didion et al., 2002; Nakazono et al., 1991). Thus, GSNOR may play additional roles in the regulation of critical organ functions. Further, nitrosative stress may contribute broadly to disease pathogenesis, since studies in endotoxemia and bacteremia are paradigmatic of other innate immune, inflammatory, degenerative and proliferative conditions in which iNOS is implicated. Thus, diseases characterized by malfunction in S-nitrosylation represent new therapeutic opportunities and targets for intervention (see Liu et al., 2004, Cell 116:617-628).

### Example 4

### Cardiac Studies

Effect of NO/SNOs on GRK2-Mediated Phosphorylation of the β₂-AR and a Soluble Peptide Substrate Using Purified Protein in a Reconstituted System

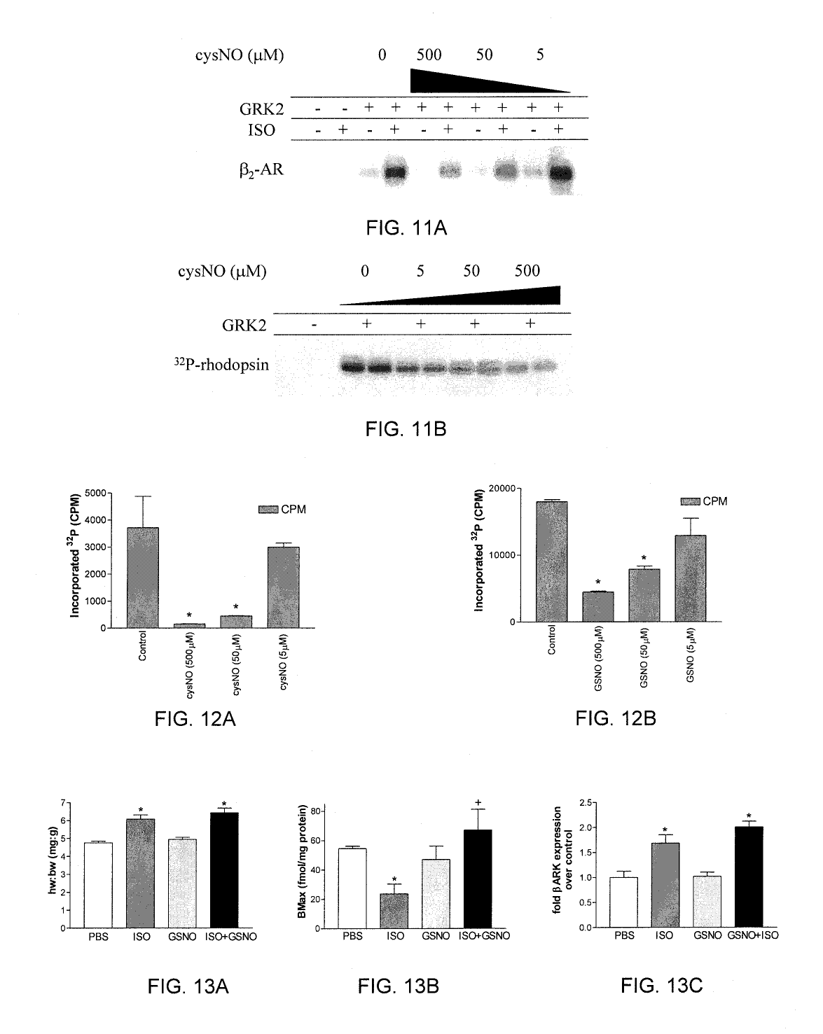

Previous data provided strong evidence supporting the hypothesis that NO prevented GRK (G protein-coupled receptor kinase)-mediated phosphorylation of the β₂-AR (adrenergic receptor). Further experiments were required to determine whether NO was acting directly on the receptor or the GRKs. To elucidate the site of NO action, its effect was tested on GRK2-mediated phosphorylation of the β₂-AR and on a soluble peptide substrate using purified proteins in a reconstituted system. In these studies, cysNO significantly decreased GRK2-mediated phosphorylation of the purified β₂-AR (FIG. 11A). NO bioactivity also significantly decreased GRK2-mediated phosphorylation of rhodopsin from purified bovine rod outer segments suggesting a generalized mechanism of NO action (FIG. 11B).

These data were in agreement with whole cell receptor phosphorylation data and limited the possible site of NO action to the receptor or the GRK. These experiments also demonstrated that SNO decreased GRK2 autophosphorylation, suggesting the direct inhibition of GRK2 by nitrosylation. To confirm this, experiments were performed to examine the capacity of SNOs to inhibit purified GRK2-mediated phosphorylation of a synthetic peptide substrate (RRREEEEESAAA; SEQ ID NO:30). In addition to decreasing GRK2-mediated receptor phosphorylation, both cysNO and GSNO significantly inhibited GRK2 mediated phosphorylation of the synthetic peptide substrate (FIG. 12A-12B).

This data provided compelling whole-cell and in vitro evidence in support of the hypothesis that NO/SNO directly decreases GRK2-mediated β₂-AR phosphorylation and provides a likely mechanism of action through S-nitrosylation of GRK2. Without being bound by theory, it was hypothesized that NO targets cysteine thiol and transition metal centers and transduces a panoply of effects, including cGMP-independent effects on many receptors by S-nitrosylation. In addition to these studies, a number of in vivo and ex vivo experiments were conducted aimed at elucidating the effects of NO on β-AR physiology and on the observed defects in β-AR function associated with heart failure. Evidence showing the involvement of S-nitrosothiols was obtained.

Effect of Bioavailable NO/Nitrosothiols on Chronic β-AR Stimulation Induced Cardiac Hypertrophy and Receptor Down-Regulation

Previous studies have demonstrated β-AR desensitization and down-regulation associated with cardiac hypertrophy and heart failure. One well established model of experimental cardiac hypertrophy in the mouse involves the chronic administration of the β-AR agonist, isoproterenol. This treatment leads to a functional uncoupling and down-regulation of cardiac β-ARs and the development of a significant increase in wall mass. Experiments were performed to study the effects of GSNO on the development of cardiac hypertrophy and its ability to alter the pattern of β-AR down-regulation associated with chronic isoproterenol stimulation.

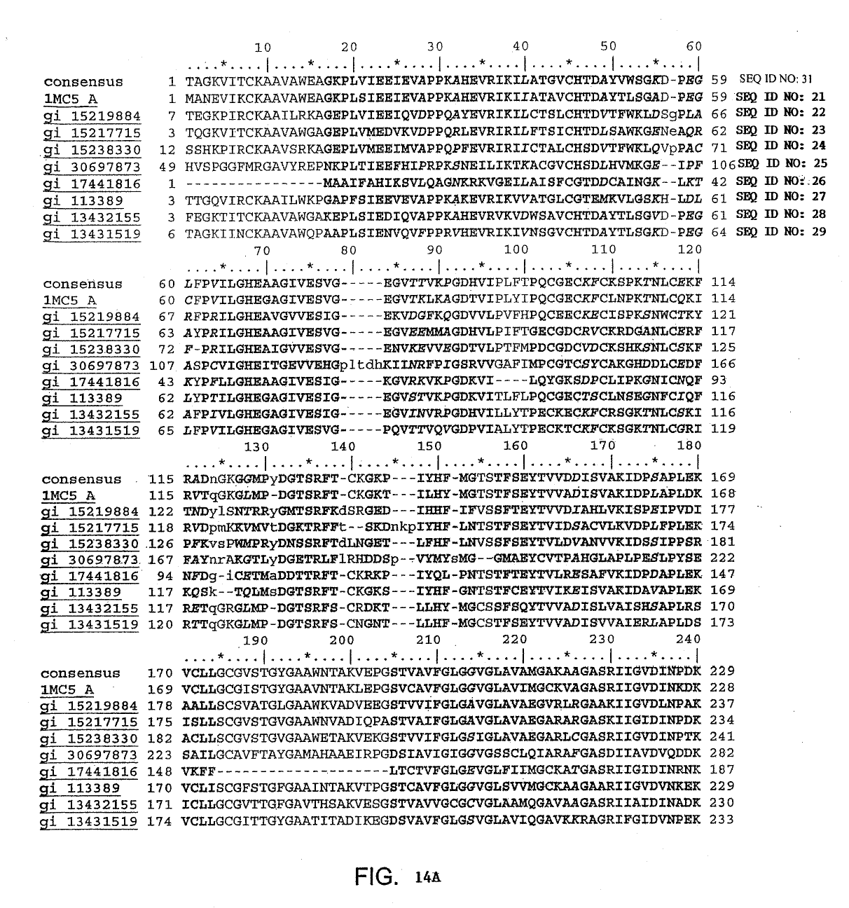

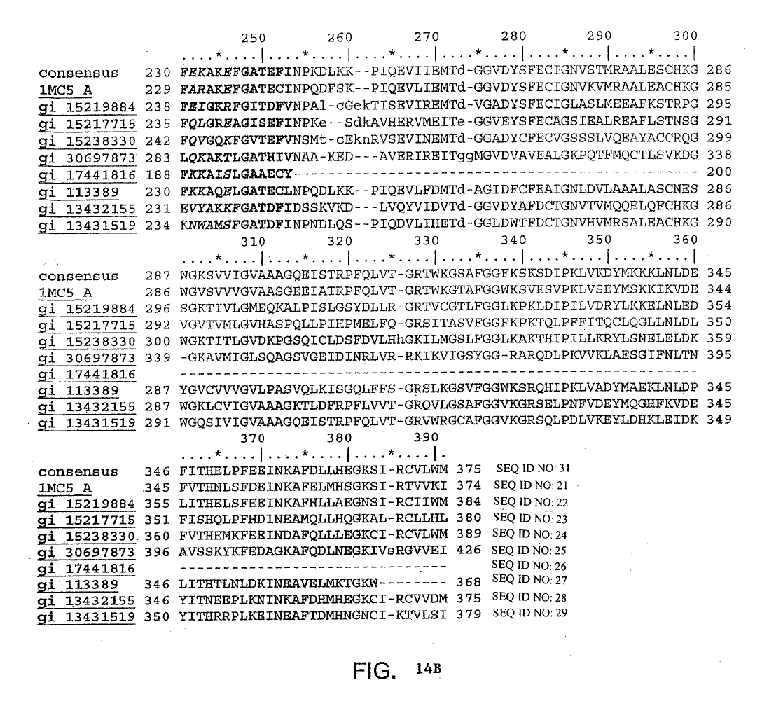

Whole cell and membrane receptor/ligand binding was performed to assess functional affinity of receptor for ligand and overall receptor density as described previously. In particular, whole cell and membrane binding was assessed for cells treated with multiple concentrations of NO/SNO in the presence and absence of desensitizing conditions (agonist pre-stimulation). The administration of GSNO (delivered via osmotic mini-pump over 2 weeks) had no effect on isoproterenol-stimulated changes in heart weight to body weight ratio (FIG. 13A), but significantly decreased β-AR down-regulation (FIG. 13B). Previous studies have demonstrated that preserving β-AR function in heart failure can delay the progression of disease. Thus, these data suggest that GSNO, by virtue of its ability to prevent the down-regulation of cardiac β-ARs, represents a novel therapeutic modality for the treatment of heart failure.

These sum of these results indicate that GSNO-elevating agents (i.e. inhibitors of GSNOR) may be used as a means to improve 62 -adrenergic signaling. FIGS. 13A-13C demonstrate that the β-adrenergic agonist isoproterenol (ISO), infused for 7 days into mice using a pump, lead to increases in cardiac weight (FIG. 13A), decreased β adrenergic receptor levels (FIG. 13B), and increased activity βARK (GRK2) expression. In contrast, the combined infusion of GSNO with ISO maintained β receptor density (FIG. 13B) because it inhibits βARK (GRK2) (FIGS. 12A-12B). Therefore, inhibitors of GSNOR alone or in combination with β-agonists could be used to improve heart failure, or other vascular disorders such as hypertension and asthma.

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims.

In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. Unless expressly stated otherwise, the techniques employed or contemplated herein are standard methodologies well known to one of ordinary skill in the art. All patents and publications cited in this specification are hereby incorporated by reference herein, including the previous disclosure provided by U.S. Application Ser. No. 60/476,055 filed Jun. 4, 2003 and U.S. Application Ser. No. 60/545,965 filed Feb. 18, 2004, and U.S. Application Ser. No. 60/550,833 filed Mar. 4, 2004.

### REFERENCES

American College of Chest Physicians/Society of Critical Care Medicine Consensus Conference: definitions for sepsis and organ failure and guidelines for the use of innovative therapies in sepsis. (1992). Crit Care Med 20, 864-874.
Boehning, D., and Snyder, S. H. (2003). Novel neural modulators. Annu Rev Neurosci 26, 105-131.
Cannon R O, 3d, Schechter A N, Panza J A, Ognibene F P, Pease-Fye M E, Waclawiw M A, Shelhamer J H, Gladwin M T. Effects of inhaled nitric oxide on regional blood flow are consistent with intravascular nitric oxide delivery. J Clin Invest. 2001;108:279-287.
Chan N L, Kavanaugh J S, Rogers P H, Arnone A. Crystallographic analysis of the interaction of nitric oxide with quaternary-T human hemoglobin. Biochemistry. 2004;43:118-132.
Cirillo, M., Laurenzi, M., Trevisan, M., and Stamler, J. (1992). Hematocrit, blood pressure, and hypertension. The Gubbio Population Study. Hypertension 20, 319-326.
Cohen, J. (2002). The immunopathogenesis of sepsis. Nature 420, 885-891.
Cosby K, Partovi K S, Crawford J H, Patel R P, Reiter C D, Martyr S, Yang B K, Waclawiw M A, Zalos G, Xu X, Huang K T, Shields H, Kim-Shapiro D B, Schechter A N, Cannon R O, 3rd, Gladwin M T. Nitrite reduction to nitric oxide by deoxyhemoglobin vasodilates the human circulation. Nat Med. 2003;9:1498-1505.
Datta B, Tufnell-Barrett T, Bleasdale R, Jones C, Beeton I, Paul V, Frenneaux M, James P. Red blood cell nitric oxide as an endocirine vasoregulator: a potential role in congestive heart failure. Circulation. in press.
De Groote, M. A., Testerman, T., Xu, Y., Stauffer, G., and Fang, F. C. (1996). Homocysteine antagonism of nitric oxide-related cytostasis in Salmonella typhimurium. Science 272, 414-417.
de Jesus-Berrios, M., Liu, L., Nussbaum, J. C., Cox, G. M., Stamler, J. S., and Heitman, J. (2003). Enzymes that counteract nitrosative stress promote fungal virulence. Curr Biol 13, 1963-1968.
Didion, S. P., Ryan, M. J., Didion, L. A., Fegan, P. E., Sigmund, C. D., and Faraci, F. M. (2002). Increased superoxide and vascular dysfunction in CuZnSOD-deficient mice. Circ Res 91, 938-944.
Eu, J. P., Liu, L., Zeng, M., and Stamler, J. S. (2000). An apoptotic model for nitrosative stress. Biochemistry 39, 1040-1047.
Feihl, F., Waeber, B., and Liaudet, L. (2001). Is nitric oxide overproduction the target of choice for the management of septic shock? Pharmacol Ther 91, 179-213.
Foglio, M. H., and Duester, G. (1996). Characterization of the functional gene encoding mouse class III alcohol dehydrogenase (glutathione-dependent formaldehyde dehydrogenase) and an unexpressed processed pseudogene with an intact open reading frame. Eur J Biochem 237, 496-504.
Foster, M. W., McMahon, T. J., and Stamler, J. S. (2003). S-nitrosylation in health and disease. Trends Mol Med 9, 160-168.
Gaston, B., Reilly, J., Drazen, J. M., Fackler, J., Ramdev, P., Arnelle, D., Mullins, M. E., Sugarbaker, D. J., Chee, C., Singel, D. J., and et al. (1993). Endogenous nitrogen oxides and bronchodilator S-nitrosothiols in human airways. Proc Natl Acad Sci USA 90, 10957-10961.
Gonzalez-Alonso, J., Olsen, D. B., and Saltin, B. (2002). Erythrocyte and the regulation of human skeletal muscle blood flow and oxygen delivery: role of circulating ATP. Circ Res 91, 1046-1055.
Gow A J, Stamler J S. Reactions between nitric oxide and haemoglobin under physiological conditions. Nature. 1998;391:169-173.
Gow, A. J., Chen, Q., Hess, D. T., Day, B. J., Ischiropoulos, H., and Stamler, J. S. (2002). Basal and stimulated protein S-nitrosylation in multiple cell types and tissues. J Biol Chem 277, 9637-9640.
Gow, A. J., Luchsinger, B. P., Pawloski, J. R., Singel, D. J., and Stamler, J. S. (1999). The oxyhemoglobin reaction of nitric oxide. Proc Natl Acad Sci USA 96, 9027-9032.
Hallstrom S, Gasser H, Neumayer C, Fugl A, Nanobashvili J, Jakubowski A, Huk I, Schlag G, Malinski T. S-nitroso human serum albumin treatment reduces ischemia/reperfusion injury in skeletal muscle via nitric oxide release. Circulation. 2002;105:3032-3038.
Haendeler, J., Hoffmann, J., Tischler, V., Berk, B. C., Zeiher, A. M., and Dimmeler, S. (2002). Redox regulatory and anti-apoptotic functions of thioredoxin depend on S-nitrosylation at cysteine 69. Nat Cell Biol 4, 743-749.
Hausladen, A., Privalle, C. T., Keng, T., DeAngelo, J., and Stamler, J. S. (1996). Nitrosative stress: activation of the transcription factor OxyR. Cell 86, 719-729.
Hausladen, A., and Stamler, J. S. (1999). Nitrosative stress. Methods Enzymol 300, 389-395.
Inoue K, Akaike T, Miyamoto Y, Okamoto T, Sawa T, Otagiri M, Suzuki S, Yoshimura T, Maeda H. Nitrosothiol formation catalyzed by ceruloplasmin. Implication for cytoprotective mechanism in vivo. J Biol Chem. 1999;274:27069-27075.
Jaffrey, S. R., Erdjument-Bromage, H., Ferris, C. D., Tempst, P., and Snyder, S. H. (2001). Protein S-nitrosylation: a physiological signal for neuronal nitric oxide. Nat Cell Biol 3, 193-197.
Jensen, D. E., Belka, G. K., and Du Bois, G. C. (1998). S-Nitrosoglutathione is a substrate for rat alcohol dehydrogenase class III isoenzyme. Biochem J 331, 659-668.
Jia, L., Bonaventura, C., Bonaventura, J., and Stamler, J. S. (1996). S-nitrosohaemoglobin: a dynamic activity of blood involved in vascular control. Nature 380, 221-226.
Jourd'heuil, D., Gray, L., and Grisham, M. B. (2000). S-nitrosothiol formation in blood of lipopolysaccharide-treated rats. Biochem Biophys Res Commun 273, 22-26.
Kirima, K., Tsuchiya, K., Sei, H., Hasegawa, T., Shikishima, M., Motobayashi, Y., Morita, K., Yoshizumi, M., and Tamaki, T. (2003). Evaluation of systemic blood NO dynamics by EPR spectroscopy: HbNO as an endogenous index of NO. Am J Physiol Heart Circ Physiol 285, H589-596.
Kluge, I., Gutteck-Amsler, U., Zollinger, M., and Do, K. Q. (1997). S-nitrosoglutathione in rat cerebellum: identification and quantification by liquid chromatography-mass spectrometry. J Neurochem 69, 2599-2607.
Knowles, R. G., Merrett, M., Salter, M., and Moncada, S. (1990). Differential induction of brain, lung, and liver nitric oxide synthase by endotoxin in the rat. Biochem J 270, 833-836.
Krege, J. H., Hodgin, J. B., Hagaman, J. R., and Smithies, O. (1995). A noninvasive computerized tail-cuff system for measuring blood pressure in mice. Hypertension 25, 1111-1115.
Laubach, V. E., Foley, P. L., Shockey, K. S., Tribble, C. G., and Kron, I. L. (1998). Protective roles of nitric oxide and testosterone in endotoxemia: evidence from NOS-2-deficient mice. Am J Physiol 275, H2211-2218.
Laubach, V. E., Shesely, E. G., Smithies, O., and Sherman, P. A. (1995). Mice lacking inducible nitric oxide synthase are not resistant to lipopolysaccharide-induced death. Proc Natl Acad Sci USA 92, 10688-10692.
Lipton, A. J., Johnson, M. A., Macdonald, T., Lieberman, M. W., Gozal, D., and Gaston, B. (2001). S-nitrosothiols signal the ventilatory response to hypoxia. Nature 413, 171-174.

Liu, D., Pavlovic, D., Chen, M. C., Flodstrom, M., Sandler, S., and Eizirik, D. L. (2000a). Cytokines induce apoptosis in beta-cells isolated from mice lacking the inducible isoform of nitric oxide synthase (iNOS^{-/-}). Diabetes 49, 1116-1122.
Liu, L., Hausladen, A., Zeng, M., Que, L., Heitman, J., and Stamler, J. S. (2001). A metabolic enzyme for S-nitrosothiol conserved from bacteria to humans. Nature 410, 490-494.
Liu, L., Zeng, M., Hausladen, A., Heitman, J., and Stamler, J. S. (2000b). Protection from nitrosative stress by yeast flavohemoglobin. Proc Natl Acad Sci USA 97, 4672-4676.
Liu L, Yan Y, Zeng M, Zhang J, Hanes M A, Ahearn G, McMahon T J, Dickfeld T, Marshall H E, Que LG, Stamler J S. (2004) Essential roles of S-nitrosothiols in vascular homeostasis and endotoxic shock. Cell. 116:617-628.
Luchsinger, B. P., Rich, E. N., Gow, A. J., Williams, E. M., Stamler, J. S., and Singel, D. J. (2003). Routes to S-nitroso-hemoglobin formation with heme redox and preferential reactivity in the beta subunits. Proc Natl Acad Sci USA 100, 461-466.
MacMicking, J. D., Nathan, C., Horn, G., Chartrain, N., Fletcher, D. S., Trumbauer, M., Stevens, K., Xie, Q. W., Sokol, K., Hutchinson, N., and et al. (1995). Altered responses to bacterial infection and endotoxic shock in mice lacking inducible nitric oxide synthase. Cell 81, 641-650.
MacMicking, J. D., North, R. J., LaCourse, R., Mudgett, J. S., Shah, S. K., and Nathan, C. F. (1997). Identification of nitric oxide synthase as a protective locus against tuberculosis. Proc Natl Acad Sci USA 94, 5243-5248.
Mannick, J. B., Hausladen, A., Liu, L., Hess, D. T., Zeng, M., Miao, Q. X., Kane, L. S., Gow, A. J., and Stamler, J. S. (1999). Fas-induced caspase denitrosylation. Science 284, 651-654.
Marshall, H. E., and Stamler, J. S. (2002). Nitrosative stress-induced apoptosis through inhibition of NF-kappa B. J Biol Chem 277, 34223-34228.
Matsumoto, A., Comatas, K. E., Liu, L., and Stamler, J. S. (2003). Screening for nitric oxide-dependent protein-protein interactions. Science 301, 657-661.
Matsushita, K., Morrell, C. N., Cambien, B., Yang, S. X., Yamakuchi, M., Bao, C., Hara, M. R., Quick, R. A., Cao, W., O'Rourke, B., et al. (2003). Nitric oxide regulates exocytosis by S-nitrosylation of N-ethylmaleimide-sensitive factor. Cell 115, 139-150.
McCarthy T J, Dence C S, Holmberg S W, Markham J, Schuster D P, Welch M J. Inhaled [¹³N]nitric oxide: a positron emission tomography (PET) study. Nucl Med Biol. 1996; 23:773-777.
McMahon, T. J., Moon, R. E., Luschinger, B. P., Carraway, M. S., Stone, A. E., Stolp, B. W., Gow, A. J., Pawloski, J. R., Watke, P., Singel, D. J., et al. (2002). Nitric oxide in the human respiratory cycle. Nat Med 8, 711-717.
Milsom, A. B., Jones, C. J., Goodfellow, J., Frenneaux, M. P., Peters, J. R., and James, P. E. (2002). Abnormal metabolic fate of nitric oxide in Type I diabetes mellitus. Diabetologia 45, 1515-1522.
Molotkov, A., Fan, X., Deltour, L., Foglio, M. H., Martras, S., Farres, J., Pares, X., and Duester, G. (2002). Stimulation of retinoic acid production and growth by ubiquitously expressed alcohol dehydrogenase Adh3. Proc Natl Acad Sci USA 99, 5337-5342.
Moncada, S., Palmer, R. M., and Higgs, E. A. (1991). Nitric oxide: physiology, pathophysiology, and pharmacology. Pharmacol Rev 43, 109-142.
Nagababu E, Ramasamy S, Abernethy D R, Rifkind J M. Active nitric oxde produced in the red cell under hypoxic conditions by deoxyhemoglobin-mediated nitrite reduction. J Biol Chem. 2003; 278:46349-46356.
Nakazono, K., Watanabe, N., Matsuno, K., Sasaki, J., Sato, T., and Inoue, M. (1991). Does superoxide underlie the pathogenesis of hypertension? Proc Natl Acad Sci USA 88, 10045-10048.
Napoli C, Aldini G, Wallace J L, de Nigris F, Maffei R, Abete P, Bonaduce D, Condorelli G, Rengo F, Sica V, D'Armiento F P, Mignogna C, de Rosa G, Condorelli M, Lerman L O, Ignarro L J. Efficacy and age-related effects of nitric oxide-releasing aspirin on experimental restenosis. Proc Natl Acad Sci USA. 2002;99:1689-1694.
Nathan, C., and Xie, Q. W. (1994). Nitric oxide synthases: roles, tolls, and controls. Cell 78, 915-918.
Ng ESM, Jourd'heuil D, McCord J M, Hernandez D, Yasui M, Knight D, Kubes P. Enhanced S-nitroso-albumin formation from inhaled NO during ischemia/reperfusion. Circ. Res. 2004; 94:559-565.
Nicholson, S. C., Hahn, R. T., Grobmyer, S. R., Brause, J. E., Hafner, A., Potter, S., Devereux, R. B., and Nathan, C. F. (1999). Echocardiographic and survival studies in mice undergoing endotoxic shock: effects of genetic ablation of inducible nitric oxide synthase and pharmacologic antagonism of platelet-activating factor. J Surg Res 86, 198-205.
Oberholzer, A., Keel, M., Zellweger, R., Steckholzer, U., Trentz, O., and Ertel, W. (2000). Incidence of septic complications and multiple organ failure in severely injured patients is sex specific. J Trauma 48, 932-937.
Ortiz, P. A., and Garvin, J. L. (2003). Cardiovascular and renal control in NOS-deficient mouse models. Am J Physiol Regul Integr Comp Physiol 284, R628-638.
Ou, J., Carlos, T. M., Watkins, S. C., Saavedra, J. E., Keefer, L. K., Kim, Y. M., Harbrecht, B. G., and Billiar, T. R. (1997). Differential effects of nonselective nitric oxide synthase (NOS) and selective inducible NOS inhibition on hepatic necrosis, apoptosis, ICAM-1 expression, and neutrophil accumulation during endotoxemia. Nitric Oxide 1, 404-416.
Pawloski, J. R., Hess, D. T., and Stamler, J. S. (2001). Export by red blood cells of nitric oxide bioactivity. Nature 409, 622-626.
Rafikova O, Rafikov R, Nudler E. Catalysis of S-nitrosothiols formation by serum albumin: the mechanism and implication in vascular control. Proc Natl Acad Sci USA. 2002; 99:5913-5918.
Rassaf T, Bryan N S, Maloney R E, Specian V, Kelm M, Kalyanaraman B, Rodriguez J, Feelisch M. NO adducts in mammalian red blood cells: too much or too little? Nat Med. 2003; 9:481-482; author reply 482-483.
Rees, D. D., Monkhouse, J. E., Cambridge, D., and Moncada, S. (1998). Nitric oxide and the hemodynamic profile of endotoxin shock in the conscious mouse. Br J Pharmacol 124, 540-546.
Rizzo, M. A., and Piston, D. W. (2003). Regulation of beta cell glucokinase by S-nitrosylation and association with nitric oxide synthase. J Cell Biol 161, 243-248.
Rodriguez, J., Maloney, R. E., Rassaf, T., Bryan, N. S., and Feelisch, M. (2003). Chemical nature of nitric oxide storage forms in rat vascular tissue. Proc Natl Acad Sci USA 100, 336-341.
Romeo, A. A., Capobianco, J. A., and English, A. M. (2003). Superoxide dismutase targets NO from GSNO to Cysbeta93 of oxyhemoglobin in concentrated but not dilute solutions of the protein. J Am Chem Soc 125, 14370-14378.
Schrammel A, Gorren A C, Schmidt K, Pfeiffer S, Mayer B. S-nitrosation of glutathione by nitric oxide, peroxynitrite, and NO/O₂⁻. Free Radic Biol Med. 2003; 34:1078-1088.
Schroder, J., Kahlke, V., Staubach, K. H., Zabel, P., and Stuber, F. (1998). Gender differences in human sepsis. Arch Surg 133, 1200-1205.
Simon D I, Mullins M E, Jia L, Gaston B, Singel D J, Stamler J S. Polynitrosylated proteins: characterization, bioactivity, and functional consequences. Proc Natl Acad Sci USA. 1996; 93:4736-4741.
Stamler J S, Simon D I, Osborne J A, Mullins M E, Jaraki O, Michel T, Singel D J, Loscalzo J. S-nitrosylation of proteins with nitric oxide: synthesis and characterization of biologically active compounds. Proc Natl Acad Sci USA. 1992; 89:444-448.
Stamler, J. S. (1999). Nitric oxide in the cardiovascular system. Coron Artery Dis 10, 273-276.
Stamler, J. S., Jaraki, O., Osborne, J., Simon, D. I., Keaney, J., Vita, J., Singel, D., Valeri, C. R., and Loscalzo, J. (1992). Nitric oxide circulates in mammalian plasma primarily as an S-nitroso adduct of serum albumin. Proc Natl Acad Sci USA 89, 7674-7677.
Stamler, J. S., Lamas, S., and Fang, F. C. (2001). Nitrosylation. the prototypic redox-based signaling mechanism. Cell 106, 675-683.
Stamler J S, Toone E J. The decomposition of thionitrites. Curr Opin Chem Biol. 2002; 6:779-785.
Jonathan S. Stamler. S-Nitrosothiols in the Blood: Roles, Amounts, and Methods of Analysis. Circ. Res., March 2004; 94: 414-417.
Stoll, M., Cowley, A. W., Jr., Tonellato, P. J., Greene, A. S., Kaldunski, M. L., Roman, R. J., Dumas, P., Schork, N. J., Wang, Z., and Jacob, H. J. (2001). A genomic-systems biology map for cardiovascular function. Science 294, 1723-1726.
Travis, M. D., Davisson, R. L., Bates, J. N., and Lewis, S. J. (1997). Hemodynamic effects of L- and D-S-nitroso-beta,beta-dimethylcysteine in rats. Am J Physiol 273, H1493-1501.
Tyurin V A, Liu S X, Tyurina Y Y, Sussman N B, Hubel C A, Roberts J M, Taylor R N, Kagan V E. Elevated levels of S-nitrosoalbumin in preeclampsia plasma. Circ Res. 2001; 88:1210-1215.
Tybulewicz, V. L., Crawford, C. E., Jackson, P. K., Bronson, R. T., and Mulligan, R. C. (1991). Neonatal lethality and lymphopenia in mice with a homozygous disruption of the c-abl proto-oncogene. Cell 65, 1153-1163.
Uotila, L., and Koivusalo, M. (1989). Gluthatione. Chemical, Biochemical and Medical Aspects. In Coenzymes and Cofactors., D. Dolphin, ed. (New York, John Wiley & Sons), pp. 517-551.
Uotila, L., and Koivusalo, M. (1997). Expression of formaldehyde dehydrogenase and S-formylglutathione hydrolase activities in different rat tissues. Adv Exp Med Biol 414, 365-371.
Wichterman, K. A., Baue, A. E., and Chaudry, I. H. (1980). Sepsis and septic shock—a review of laboratory models and a proposal. J Surg Res 29, 189-201.
Yang, Z. N., Bosron, W. F., and Hurley, T. D. (1997). Structure of human chi chi alcohol dehydrogenase: a glutathione-dependent formaldehyde dehydrogenase. J Mol Biol 265, 330-343.
Zaman, K., McPherson, M., Vaughan, J., Hunt, J., Mendes, F., Gaston, B., and Palmer, L. A. (2001). S-nitrosoglutathione increases cystic fibrosis transmembrane regulator maturation. Biochem Biophys Res Commun 284, 65-70.
Zellweger, R., Wichmann, M. W., Ayala, A., Stein, S., DeMaso, C. M., and Chaudry, I. H. (1997). Females in proestrus state maintain splenic immune functions and tolerate sepsis better than males. Crit Care Med 25, 106-110.
Zunic, G., Spasic, S., and Jelic-Ivanovic, Z. (1999). Simple and rapid method for the measurement of nitrite and nitrate in human plasma and cerebrospinal fluid by capillary electrophoresis. J Chromatogr B Biomed Sci Appl 727, 73-79.
Sequence information was obtained from NCBI (Bethesda, Md.), online at hypertext transfer protocol://world wide web.ncbi.nlm.nih.gov/.

What is claimed is:

1. A method for alleviating or inhibiting the onset of at least one symptom of a disorder associated with increased levels of nitric oxide bioactivity comprising administering to a patient with the disorder a therapeutically effective amount of an agent that increases activity or levels of a S-nitrosoglutathione reductase and/or decreases levels of S-nitrosothiols.
